(19) United States
(12) Patent Application Publication
(10) Pub. No.: US 2026/0090937 A1
(43) Pub. Date: Apr. 2, 2026

(54) MEDICAL BED APPARATUSES, MEDICAL SYSTEMS AND MEDICAL METHODS

(71) Applicant: SHANGHAI UNITED IMAGING HEALTHCARE CO., LTD., Shanghai (CN)

(72) Inventors: Dehe LI, Shanghai (CN); Lingling LI, Shanghai (CN); Xiaobin LI, Shanghai (CN)

(73) Assignee: SHANGHAI UNITED IMAGING HEALTHCARE CO., LTD., Shanghai (CN)

(21) Appl. No.: 19/413,041

(22) Filed: Dec. 9, 2025

Related U.S. Application Data
(63) Continuation-in-part of application No. PCT/CN2024/098288, filed on Jun. 8, 2024.

(30) Foreign Application Priority Data
Jun. 9, 2023 (CN) 202310686216.7
Jun. 9, 2023 (CN) 202321472852.1
Jun. 9, 2023 (CN) 202321475921.4
Jun. 9, 2023 (CN) 202321475969.5
Aug. 25, 2025 (CN) 202521813929.6
Sep. 2, 2025 (CN) 202521889532.5

(51) Int. Cl. A61G 7/10; A61B 6/04

(52) U.S. Cl. CPC A61G 7/103; A61G 7/1001; A61G 7/1046; A61B 6/0487

(57) ABSTRACT

Embodiments of the present disclosure provide a medical bed apparatus, a medical system, and a medical method. The medical bed apparatus includes a medical bed, a movable bed board, and a conveying device. The conveying device is detachably docked with the movable bed board, and the conveying device is configured to transfer, on the medical bed, the movable bed board. The medical system includes a medical device and the medical bed apparatus.

### CROSS-REFERENCE TO RELATED APPLICATIONS

This application is a Continuation-in-part International Application No. PCT/CN2024/098288 filed on Jun. 8, 2024, which claims priority to Chinese Application No. 202310686216.7 filed on Jun. 9, 2023, Chinese Application No. 202321475921.4 filed on Jun. 9, 2023, Chinese Application No. 202321472852.1 filed on Jun. 9, 2023, and Chinese Application No. 202321475969.5 filed on Jun. 9, 2023, and this application claims priority to Chinese Application No. 202521813929.6, filed on Aug. 25, 2025, and Chinese Application No. 202521889532.5, filed on Sep. 2, 2025, the contents of each of which are hereby incorporated by reference.

### TECHNICAL FIELD

The present disclosure relates to the field of medical devices, particularly to medical bed apparatuses, medical systems, and medical methods.

### BACKGROUND

A medical device is equipped with a medical space for radiological diagnosis or treatment of the subject, and a medical bed (also called a medical table) transports the subject to the medical space to enable diagnosis or treatment of the part of the subject to be examined. However, if the axial dimension of a medical space (e.g., a circular medical space surrounding the subject) are long, this will result in a subsequent increase in the distance that the medical bed has to transfer the subject, which is likely to make it difficult for existing medical beds to transfer the subject in a stable manner. In addition, the operation of transferring a subject who has mobility problems of his or her own into a medical space can be inconvenient.

Therefore, how to conveniently and stably transfer the subject into the medical space is a technical problem that needs to be urgently solved in this field.

### SUMMARY

One or more embodiments of the present disclosure provide a medical bed apparatus.

The medical bed apparatus includes a medical bed, a movable bed board, and a conveying device. The conveying device is detachably docked with the movable bed board, and the conveying device is configured to transfer, on the medical bed, the movable bed board.

In some embodiments, the medical bed includes a medical bed board and a support device, and the support device is configured to drive the medical bed board to move in multiple degrees of freedom.

In some embodiments, the support device is configured to drive the medical bed board to move along three orthogonal directions; or the support device is configured to drive the medical bed board to move in six degrees of freedom.

In some embodiments, the support device includes a plurality of telescopic rods. For each of the plurality of telescopic rods, one end of the telescopic rod is connected with the medical bed board via a first hinge seat, and the other end of the telescopic rod is connected with a second hinge seat; and a portion of the plurality of telescopic rods is connected to one end of the medical bed board, and another portion of the plurality of telescopic rods is connected to the other end of the medical bed board.

In some embodiments, the first hinge seats are spaced apart along a first circumference, and the second hinge seats are spaced apart along a second circumference.

In some embodiments, the support device includes at least three telescopic rods. A portion of the at least three telescopic rods is connected with the one end of the medical bed board, and the rest of the at least three telescopic rods is connected with the other end of the medical bed board.

In some embodiments, the support device includes six telescopic rods, two of the six telescopic rods are connected with the one end of the medical bed board, and the remaining four of the six telescopic rods are connected with the other end of the medical bed board; or the support device includes six telescopic rods, three of the six telescopic rods are connected with the one end of the medical bed board, and the remaining three of the six telescopic rods are connected with the other end of the medical bed board.

In some embodiments, the movable bed board, as a part of a transfer bed, is detachably mounted on a movable bed body, which is another part of the transfer bed.

In some embodiments, the conveying device is disposed on the medical bed; and the conveying device is detachably docked with at least a portion of the transfer bed via a docking assembly.

In some embodiments, the docking assembly includes a first docking member, a second docking member, and a locking mechanism; one of the first docking member and the second docking member is disposed on the movable bed board, and the other of the first docking member and the second docking member is disposed on the conveying device; and the first docking member is configured to dock with the second docking member, and the locking mechanism is configured to connect and lock the first docking member and the second docking member docked with each other.

In some embodiments, the docking assembly includes a receiving plate and a gap, the receiving plate is disposed on the conveying device, the gap is located between the movable bed body and the movable bed board, and the receiving plate is capable of being inserted into the gap to support the movable bed board.

In some embodiments, the first docking member includes a docking protrusion, and the second docking member includes a docking groove; one of an outer wall of the docking protrusion and an inner wall of the docking groove is provided with a helical groove, and the other of the outer wall of the docking protrusion and the inner wall of the docking groove is provided with a protrusion part capable of being inserted into the helical groove; and the locking mechanism includes a first driving member, and the first driving member is configured to drive the docking protrusion to rotate along an axis of the helical groove, so that the protrusion part moves along the helical groove.

In some embodiments, the first docking member includes at least one hook, and the second docking member includes at least one docking groove; the at least one hook is provided on one of the conveying device and the movable bed board, and the at least one docking groove is provided on another one of the conveying device and the movable bed board; and the at least one hook is capable of being connected with or disconnected from the at least one docking groove by rotation.

In some embodiments, the medical bed apparatus further includes at least one resilient member and an unlocking member. The at least one hook is disposed on the unlocking member, and the at least one resilient member is arranged between the conveying device and the unlocking member.

In some embodiments, the medical bed apparatus further includes a drive mechanism driving the at least one hook to rotate. The drive mechanism includes a drive cable and a reel, the reel is disposed at one end of the medical bed that is far from a docking location of the conveying device and the movable bed board, and the drive cable is connected with the reel and the unlocking member.

In some embodiments, each of the at least one hook is provided with a first guiding slope, the movable bed board is provided with a guiding member, the guide member and the at least one docking groove are arranged along a conveying direction of the conveying device, and the guide member cooperates with the first guiding slope.

In some embodiments, the medical bed apparatus further includes a lifting platform. The lifting platform is disposed on the medical bed and is capable of being raised and lowered.

In some embodiments, the lifting platform includes a lifting seat, a lifting drive mechanism, and a bed frame; the bed frame is fixed to the lifting seat, the movable bed board is detachably disposed on the bed frame, and the lifting seat is connected to the medical bed; and the lifting drive mechanism is drivingly connected to the lifting seat.

One or more embodiments of the present disclosure provide a medical system. The medical system includes a medical device and a medical bed apparatus described above. The medical device includes a medical space, a medical bed board of the medical bed of the medical bed apparatus is configured to pass through the medical space along an axial direction of the medical space.

One or more embodiments of the present disclosure provide a medical method using the medical system described above for medical therapy or medical imaging. The medical method includes: transferring the movable bed board through the conveying device of the medical bed apparatus; placing the movable bed board onto the medical bed board of the medical bed of the medical bed apparatus at a position within the medical space of the medical device; adjusting a position of a subject by driving, using a support device of the medical bed, the medical bed board to move; and performing a medical operation on the subject using the medical device.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating a structure of a medical device and a medical bed according to some embodiments of the present disclosure;

FIG. 2 is a schematic diagram illustrating a structure of a medical bed apparatus according to some embodiments of the present disclosure;

FIG. 3 is a schematic diagram of a use of a medical bed apparatus according to some embodiments of the present disclosure;

FIG. 4 is a schematic diagram of a use of a medical bed apparatus according to some embodiments of the present disclosure;

FIG. 5 is a schematic diagram illustrating a structure of a docking assembly according to some embodiments of the present disclosure;

FIG. 6 is an enlarged schematic diagram at M of FIG. 5;

FIG. 7 is a schematic diagram illustrating a structure of a docking protrusion and a first driving member of a docking assembly according to some embodiments of the present disclosure;

FIG. 8 is a schematic diagram illustrating a structure of a docking assembly according to some other embodiments of the present disclosure;

FIG. 9 is a schematic diagram illustrating a structure of a docking assembly according to some other embodiments of the present disclosure;

FIG. 10 is a schematic diagram illustrating a structure of a docking assembly according to yet some more embodiments of the present disclosure;

FIG. 11 is a schematic diagram illustrating a structure of a conveying device in the embodiment of FIG. 10;

FIG. 12 is an enlarged schematic diagram of part D of FIG. 10;

FIG. 13 is an exemplary schematic diagram illustrating a structure of a medical bed apparatus according to some embodiments of the present disclosure;

FIG. 14 is an exemplary schematic diagram of a hook connected with a docking groove according to some embodiments of the present disclosure;

FIG. 15 is a schematic diagram illustrating a localized structure of a movable bed board according to some embodiments of the present disclosure;

FIG. 16 is an exemplary schematic diagram illustrating a structure of a hook disconnected from a docking groove according to some embodiments of the present disclosure;

FIG. 17 is an exemplary schematic diagram illustrating a structure of a movable bed board disconnected from a conveying device according to some embodiments of the present disclosure;

FIG. 18 is a schematic diagram illustrating a structure of a knob-type locking docking mechanism according to some embodiments of the present disclosure;

FIG. 19 is a schematic diagram illustrating a structure of a docking assembly according to some further embodiments of the present disclosure;

FIG. 20 is a schematic diagram illustrating a structure of a docking assembly according to yet other embodiments of the present disclosure;

FIG. 21 is a schematic diagram illustrating a structure of a docking assembly according to yet other embodiments of the present disclosure;

FIG. 22 is a schematic diagram illustrating a structure of a medical bed according to some embodiments of the present disclosure;

FIG. 23 is a schematic diagram illustrating a structure of ae medical bed according to some embodiments of the present disclosure;

FIG. 24 is a schematic diagram illustrating a structure of a medical bed according to some embodiments of the present disclosure;

FIG. 25 is a schematic diagram illustrating a structure of a first circumference and a second circumference according to some embodiments of the present disclosure;

FIG. 26 is a schematic diagram illustrating a structure of a conveying device according to some embodiments of the present disclosure;

FIG. 27 is a schematic diagram illustrating a structure of a medical system according to some embodiments of the present disclosure;

FIG. 28 is a schematic diagram illustrating a structure of a medical bed apparatus according to some embodiments of the present disclosure;

FIG. 29 is a schematic diagram illustrating a structure of a medical bed apparatus according to some embodiments of the present disclosure;

FIG. 30 is a schematic diagram illustrating a structure of a lifting platform with a docking assembly according to some embodiments of the present disclosure;

FIG. 31 is a schematic diagram illustrating a structure of a lifting platform according to some embodiments of the present disclosure;

FIG. 32 is a schematic diagram illustrating a structure of a bed plate locking mechanism and a bed plate limiting mechanism according to some embodiments of the present disclosure;

FIG. 33 is a schematic diagram illustrating an internal structure of a bed plate locking mechanism according to some embodiments of the present disclosure;

FIG. 34 is a schematic diagram illustrating a structure of a bed plate limiting mechanism according to some embodiments of the present disclosure; and

FIG. 35 is a schematic diagram of a use of a medical system according to some embodiments of the present disclosure.

### DETAILED DESCRIPTION

In order to more clearly illustrate the technical solutions of the embodiments of the present disclosure, the accompanying drawings to be used in the description of the embodiments will be briefly described below. Obviously, the accompanying drawings in the following descriptions are only some examples or embodiments of the present disclosure, and that the present disclosure may be applied to other similar scenarios in accordance with these drawings without creative labor for those of ordinary skill in the art. Unless obviously acquired from the context or the context illustrates otherwise, the same numeral in the drawings refers to the same structure or operation.

As indicated in the present disclosure and in the claims, the singular forms “a,” “an,” and “the” may be intended to include the plural forms as well, unless the context clearly indicates otherwise. In general, the terms “comprise,” “comprises,” and/or “comprising,” “include,” “includes,” and/or “including,” when used in this disclosure, specify the presence of stated features, integers, steps, operations, elements, and/or components, but do not preclude the presence or addition of one or more other features, integers, steps, operations, elements, components, and/or groups thereof.

The present disclosure provides a medical bed apparatus, which may be used in various medical systems. In some embodiments, the medical bed apparatus may be used in scenarios with long boreholes and requiring multi-degree-of-freedom rotation. Exemplary application scenarios may include a radiotherapy system guided by a multimodal imaging device (e.g., a Positron Emission Tomography□Magnetic Resonance-Radiotherapy (PET-MR-RT) device). However, the medical bed apparatus is not limited to the exemplary application scenarios. In some embodiments, the medical bed apparatus may be used in multimodal imaging devices (e.g., a Positron Emission Tomography-Computed Tomography (PET-CT) device, a Positron Emission Tomography-Magnetic Resonance-Computed Tomography (PET-MR-CT) device, etc.), single-modality imaging-guided radiotherapy systems (e.g., a Magnetic Resonance Radiotherapy (MR-RT) device, a Positron Emission Tomography-Radiotherapy (PET-RT) device, etc.), or the like. In some embodiments, the medical bed apparatus may be used independently as needed.

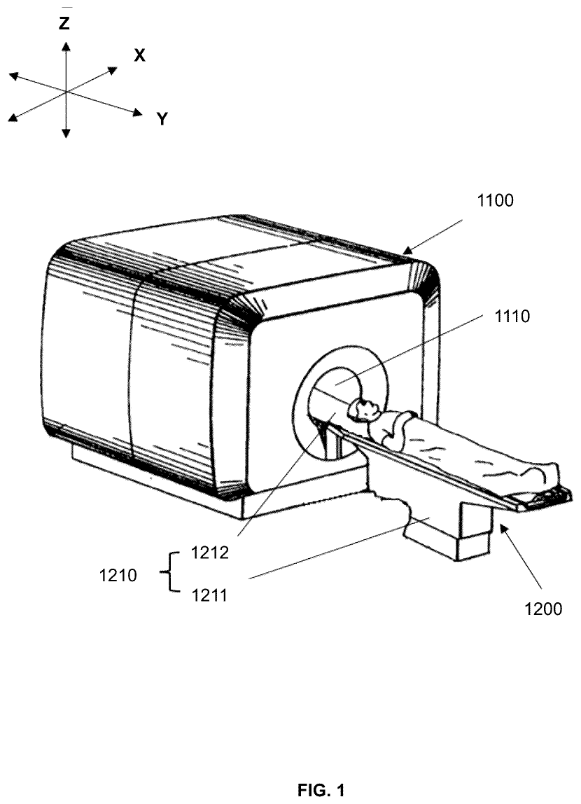

FIG. 1 is a schematic diagram illustrating a structure of a medical device and a medical bed according to some embodiments of the present disclosure. As shown in FIG. 1, a medical system may include a medical device 1100 and a medical table 1200. The medical device 1100 may include a medical space 1110 for radiographic diagnosis or treatment of a subject. The medical space 1110 shown in FIG. 1 is a circular medical space 1110 surrounding the subject.

The medical table 1200 may be used to transport the subject into the medical space 1110. For the circular medical space 1110, the space available for setting up the medical table 1200 may be relatively limited.

In some embodiments, as shown in FIG. 1, the medical table 1200 may be a cantilevered medical table 1210. The cantilevered medical table 1210 may include a cantilevered base 1211 and a cantilevered bed plate 1212. The cantilevered base 1211 may be positioned at one end of an axial direction (e.g., a Y direction in FIG. 1) of the circular medical space 1110, and the cantilevered bed plate 1212 may move relative to the cantilevered base 1211 along the axial direction of the circular medical space 1110 to transport the subject into the medical space 1110. Since the cantilevered base 1211 of the cantilevered medical table 1210 is positioned at one end of the axial direction (i.e., the Y direction in FIG. 1) of the medical space 1110, if an axial dimension of the medical space 1110 is relatively long, a transport distance of the cantilevered bed plate 1212 also needs to be extended, leading to unstable transportation of the cantilevered medical table 1210. For example, due to the lack of support at the end of the cantilevered bed plate 1212 away from the cantilevered base 1211, the cantilevered bed plate 1212 may bend. Additionally, for some subjects with limited mobility in the medical system, transferring the subjects onto the medical table 1200 may be very inconvenient.

In view of the foregoing, one or more embodiments of the present disclosure provide the medical bed apparatus, which may include at least one of a medical bed, a transfer bed, and a conveying device. At least a portion of the medical bed may be disposed within a medical space of the medical device, and the medical bed may be subject to multiple degrees of freedom (e.g., six degrees of freedom) movement. The subject may be placed on the medical bed for imaging or radiation therapy, and the transfer bed may be configured to transfer the subject near the medical bed or the conveying device.

In some embodiments, the medical bed apparatus may include the medical bed and the conveying device. In some embodiments, the medical bed apparatus may include the medical bed, the movable bed board, and the conveying device.

By providing a support device to support both ends of the medical bed board, the stability of the medical bed board can be ensured even if the axial dimension of the medical space is long. By the cooperation of the medical bed, the transfer bed, and the conveying device can realize the convenient transfer of the movable bed board, and even in the scenario where the medical space is narrower or the axial length is longer, the medical operation can be conveniently carried out.

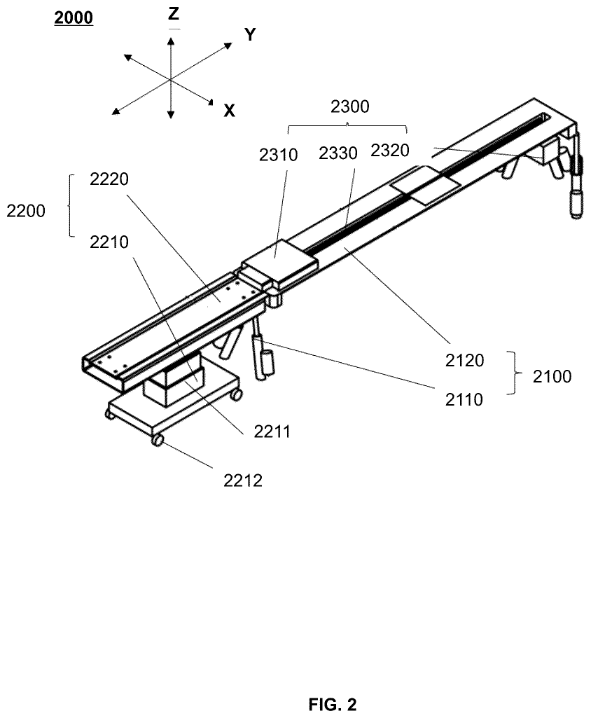

FIG. 2 is a schematic diagram illustrating a structure of a medical bed apparatus according to some embodiments of the present disclosure. As shown in FIG. 2, a medical bed apparatus 2000 includes a medical bed 2100, a movable bed board 2220, and a conveying device 2300.

The medical bed 2100 refers to a bed configured to support a patient during a medical procedure, at least a portion of which may be disposed within the medical space 1110 of a medical system so that a patient on the medical bed 2100 may receive a diagnosis or treatment within the medical space 1110. The movable bed board 2220 refers to a movable plate-like structure configured to support a patient during a medical procedure.

The conveying device 2300 is configured to transfer the subject (i.e., the patient). In some embodiments, the conveying device 2300 is removably connected to the movable bed board 2220, and the conveying device 2300 is configured to transfer the movable bed board 2220 on the medical bed 2100, thereby transferring the subject. In some embodiments, the movable bed board 2220 is made of a high-strength lightweight material (e.g., high-density fiberboard, aluminum alloy, etc.) with sufficient strength and stability to ensure the safety of the subject.

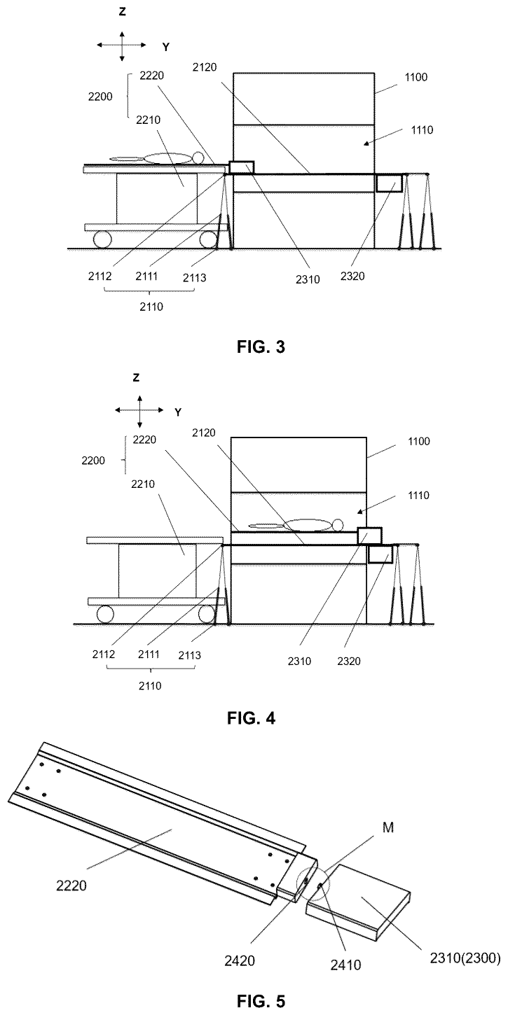

In some embodiments of the present disclosure, at least a portion of the medical bed 2100 is disposed within the medical space 1110 of the medical device (as shown in FIGS. 3-4 and 19-21). The medical bed 2100 may include a medical bed board 2120 and a support device 2110 that supports both ends of the medical bed board 2120 along a length of the medical bed board 2120, and the support device 2110 is configured to actuate the medical bed board 2120 to move in multiple degrees of freedom. In some embodiments, the support device 2110 may drive the medical bed board 2120 in at least three orthogonal directions.

The medical bed board 2120 refers to a platform for carrying a subject, and the support device 2110 may provide stabilizing support for the medical bed board 2120. In some embodiments, the support device 2110 may be disposed at both ends of the medical bed board 2120.

The conveying device 2300 is configured to transfer the movable bed board 2220. The conveying device 2300 may be detachably docked with the movable bed board 2220, and may transport the movable bed board 2220 onto the medical bed board 2120, further, the conveying device 2300 may transport the movable bed board 2220 to a position of the medical bed board 2120 located within the medical space 1110 of the medical device. More descriptions regarding the conveying device 2300 may be found elsewhere in FIGS. 3-4 and 10, and related descriptions thereof.

In some embodiments, at least a portion of the medical bed 2100 is disposed within the medical space 1110 of the medical system to enable a subject on the medical bed 2100 to be seen and treated within the medical space 1110.

The support device 2110 may drive the medical bed board 2120 to move, thereby adjusting a position of the subject for easier radiographic diagnosis and/or treatment operations. The support device 2110 may drive the medical bed board 2120 to move in multiple degrees of freedom.

In some embodiments, the support device 2110 may drive the medical bed board 2120 to move in three orthogonal directions. In some embodiments, the support device 2110 may drive the medical bed board 2120 to move in six degrees of freedom.

The support device 2110 may drive the medical bed board 2120 to translate along one direction and/or rotate around the direction, or the support device 2110 may drive the medical bed board 2120 to translate along at least two different directions and/or rotate around the at least two different directions to achieve motion of the medical bed board 2120 in multiple degrees of freedom.

In some embodiments, the support device 2110 is capable of driving the medical bed board 2120 to translate along and/or rotate around the three different directions.

In some embodiments, the support device 2110 may drive the medical bed board 2120 to move (e.g., translate, rotate) along the three orthogonal directions. For example, the support device 2110 may drive the medical bed board 2120 to translate or rotate along an X-axis direction, a Y-axis direction, and a Z-axis direction shown in FIG. 2. As another example, the support device 2110 may drive the medical bed board 2120 to translate along one or more directions of the X-axis direction, Y-axis direction, and Z-axis direction and rotate around another one or more directions of the X-axis direction, Y-axis direction, and Z-axis direction. As yet another example, the support device 2110 may drive the medical bed board 2120 to translate along and rotate around the X-axis direction, Y-axis direction, and Z-axis direction shown in FIG. 2. More descriptions for the specific structure of the support device 2110 may be found in FIGS. 21-22 and 24 and related descriptions thereof.

In some embodiments, the support device 2110 may drive the medical bed board 2120 to move in multiple degrees of freedom simultaneously (e.g., six degrees of freedom simultaneously). In other embodiments, the support device 2110 may drive the medical bed board 2120 to first perform one or more degrees of freedom of movement, and then drive the medical bed board 2120 to perform an additional one or more degrees of freedom of movement to position the subject to a suitable position. In other words, the movement of the medical bed board 2120 in multiple degrees of freedom may occur simultaneously or independently in an order among different degrees of freedom.

In some embodiments, the movable bed board 2220, as a first part of a transfer bed 2200, is detachably mounted on a movable bed body 2210 which is a second part of the transfer bed 2200 The transfer bed 2200 may be configured to transfer a subject. For example, the transfer bed 2200 may be configured to transfer the subject from a hospital room, an outpatient clinic, an operating room, or the like, to a space in which the medical system is located. The movable bed board 2220 being removably mounted on the movable bed body 2210 may be understood as follows: the movable bed board 2220 is provided on the movable bed body 2210, and the movable bed board 2220 may be removed from the movable bed body 2210. For example, the movable bed board 2220 may be coupled to the movable bed body 2210 in a removably coupled manner. As another example, the movable bed board 2220 may be placed directly on the movable bed body 2210 (the two may not be connected). When the movable bed board 2220 is placed on the movable bed body 2210, the movable bed board 2220 may be removed from the movable bed body 2210 in one or more directions (e.g., lifting the movable bed board 2220 upward may make it be removed from the movable bed body 2210□The conveying device 2300 is configured to transfer a subject between transfer bed 2200 and medical bed board 2120. In some embodiments, the conveying device 2300 may transfer a subject from the transfer bed 2200 to the medical bed 2100, and/or move the bed to a different location on the medical bed 2100. In some embodiments, the conveying device 2300 may be removably docked with at least a portion of the transfer bed 2200 so as to drive that portion of the transfer bed 2200 along with the transferring of the subject.

In some embodiments, the transfer bed 2200 may be any feasible structure for placing the subject, such as a general hospital bed. A transfer bed in the broadest sense may not be a bed structure. For example, the transfer bed in the broadest sense may be a wheelchair structure. As another example, the transfer bed in the broadest sense may be a plate-like structural object. In some embodiments, the transfer bed 2200 may be considered part of the conveying device 2300 or the medical bed board 2120. In other embodiments of the present disclosure, the transfer bed 2200 may be non-required, e.g., when the subject has good mobility, the subject may move to the medical bed board 2120 on his or her own without the use of a transfer bed 2200.

The conveying device 2300 is configured to transfer the movable bed board 2220. In some embodiments, the conveying device 2300 transports the movable bed board 2220 that has detached from the movable bed body 2210 to a position on the medical bed board 2120 located within the medical space 1110 of the medical system, to facilitate the examination and treatment of the subject. The conveying device 2300 may adjust the location of the movable bed board 2220 within the medical space 1110 based on a variety of factors, such as a type of medical operations specifically to be performed by the subject, an area of the subject to be treated, and a body size of the subject.

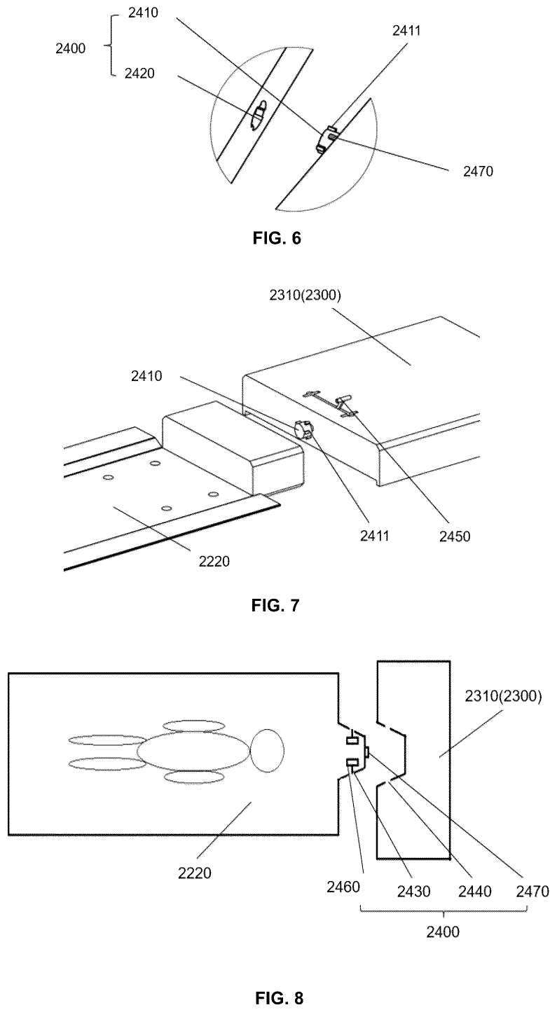

In some embodiments, the medical bed apparatus 2000 may transport the subject as follows: the medical bed apparatus 2000 may transfer the subject (from a ward, an outpatient room, an operating room, etc.) to a position near the conveying device 2300 (e.g., a location where the conveying device 2300 may dock) via the transfer bed 2200. The conveying device 2300 may be docked with the movable bed board 2220 via a docking assembly 2400 (as shown in FIG. 6). The movable bed board 2220 may be detached from the movable bed body 2210, the conveying device 2300 may further transfer the movable bed board 2220, and subsequently the movable bed board 2220 may be transported to the medical bed 2100. In some embodiments, the conveying device 2300 may detach from the movable bed board 2220 first, and after a diagnostic and medical operation is completed, the conveying device 2300 may re-dock with the movable bed board 2220 and transport the movable bed board 2220 back to the movable bed body 2210. In other embodiments, the conveying device 2300 may remain connected to the movable bed board 2220 at all times during the medical operation and convey the movable bed board 2220 back to the movable bed body 2210 when the medical operation is completed.

By providing the support devices 2110 at both ends of the medical bed board 2120, the support devices 2110 can provide stable support for the medical bed board 2120 even if the axial (along the Y-axis direction) dimension of the medical space 1110 is long. At the same time, the support device 2110 may also drive the medical bed board 2120 in motion to adjust the position of the patient for carrying out the diagnosis and treatment operation. The cooperation of the medical bed 2100, the transfer bed 2200, and the conveying device 2300 can realize the convenient transfer of the movable bed board 2220, so that even in the narrow medical space 1110 or the axial length of the long medical space 1110, the radiological diagnosis and treatment of a patient can be conveniently performed.

In some embodiments, the conveying device 2300 may be located adjacent to the medical bed 2100, e.g., above the medical bed 2100. Merely by way of example, the conveying device 2300 may include a telescopic transfer rod (not shown in the drawings) that may be located above the medical bed 2100 and extend along a length direction of the medical bed 2100. The movable bed board 2220 may be connected to one end of the telescopic transfer rod, and by telescoping the telescopic transfer rod, the movable bed board 2220 may be driven along the length of the medical bed 2100 to move the movable bed board 2220 from the movable bed body 2210 to the medical bed 2100.

In some embodiments, if the space around the medical bed 2100 is limited (e.g., by the medical space 1110 of the medical device 1100), the conveying device 2300 may be provided on the medical bed 2100.

FIG. 3 is a schematic diagram of the use of the medical bed apparatus according to some embodiments of the present disclosure. FIG. 4 is a schematic diagram of the use of the medical bed apparatus according to some embodiments of the present disclosure. In some embodiments of the present disclosure, a conveying device 2300 is provided on a medical bed 2100 as shown in FIGS. 3-4. The conveying device 2300 may include a transfer member 2310 and a drive device 2320 for driving the transfer member 2310. FIGS. 3-4 illustrate the medical bed apparatus 2000 operating in the medical system. The drive device 2320 may drive the transfer member 2310 over the medical bed board 2120 of the medical bed 2100 along a length of the medical bed 2100.

In some embodiments of the present disclosure, the conveying device 2300 is removably connected to at least a portion of the transfer bed 2200 via a docking assembly 2400.

The docking assembly 2400 may be provided on the transfer member 2310 and the movable bed board 2220. The movable bed body 2210 may drive the movable bed board 2220 to move to one end of the medical bed 2100, the transfer member 2310 and the movable bed board 2220 are detachably connected by the docking assembly 2400, and the drive device 2320 may drive the transfer member 2310 to drive the movable bed board 2220 along the length direction of the medical bed 2100, positioning the movable bed board 2220 to a position suitable for a diagnostic and treatment operation.

Referring further to FIG. 2, in some embodiments, a guide structure 2330 (e.g., a guide rail extending along the length direction of the medical bed 2100) may be provided on the medical bed 2100. The transfer member 2310 may move along the guide structure 2330 to ensure stable movement of the transfer member 2310 along the length direction of the medical bed 2100.

In some embodiments, the medical bed apparatus 2000 further includes a plurality of distributed gravity sensing devices and a speed control device. The plurality of distributed gravity sensing devices may be configured to detect gravity distribution data or pressure distribution data of a plurality of preset points on the medical bed 2100. The plurality of distributed gravity sensing devices may be distributed at the plurality of preset points on the medical bed 2100, and may be configured to obtain the gravity distribution data. The speed control device may be disposed on the conveying device 2300 for adjusting the transmission speed of the conveying device 2300 based on the gravity distribution data.

In some embodiments, the plurality of distributed gravity sensing devices may include a plurality of pressure sensors distributed at different locations.

The preset points are pre-selected locations on the medical bed 2100, which may be set according to actual needs. In some embodiments, the preset points may be distributed at two ends and/or two sides of the medical bed 2100 along the length direction of the medical bed 2100. In some embodiments, a count of preset points set at the two ends of the medical bed 2100 may be greater than a count of preset points set at other locations of the medical bed 2100.

The gravity distribution data refers to data related to the gravity detected by the plurality of distributed gravity sensing devices. In some embodiments, the gravity distribution data may include at least one of a gravity magnitude, a gravity distribution position, etc., detected by the plurality of distributed gravity sensing devices.

In some embodiments, when the movable bed board 2220 moves on the medical bed 2100, it covers the plurality of distributed gravity sensing devices or a portion thereof. The covered distributed gravity sensing devices bear a pressure of the movable bed board 2220, and the pressure borne at the preset points may be detected, which corresponds to the gravity magnitude of the corresponding part of the movable bed board 2220. The distribution positions of the plurality of distributed gravity sensing devices may represent the gravity distribution positions.

In some embodiments, the distributed gravity sensing devices may communicate with the speed control device.

The speed control device may analyze and process the gravity distribution data, and generate a corresponding control instruction, which may be sent to the conveying device 2300 to adjust the transmission speed of the conveying device 2300.

The transmission speed refers to the transfer speed of the transfer member 2310. The speed control device may adjust at least one of a rotational speed, a power, or the like. In some embodiments, the speed control device may include at least one of a variable speed motor, a transmission chain with an adjustable gear ratio, etc. The transmission chain may include at least one of a gear transmission chain, a belt transmission chain, a chain transmission chain, etc. In some embodiments, if a variable speed motor is used as the speed control device, it may replace the drive device 2320. In some embodiments, if a transmission chain with an adjustable gear ratio is used in the speed control device, it may be connected between the drive device 2320 and the transfer member 2310 in a transmission way.

By monitoring pressures at the plurality of preset points of the medical bed 2100 in real time through the plurality of distributed gravity sensing devices, it is possible to analyze whether the forces at the multiple preset points and the support forces are equal. When the support force is less than the force, the transmission speed may be reduced to prevent instability such as fluctuations during transmission

In some embodiments, through the gravity distribution data, it is possible to determine whether the gravity at the multiple preset points of the medical bed 2100 is evenly distributed. For example, the gravity magnitude of each preset point in the gravity distribution data may be compared with a preset gravity threshold corresponding to the gravity magnitude. If the gravity magnitude is less than or equal to the preset gravity threshold, it may be determined that the gravity distribution is even. If the gravity magnitude is greater than the preset gravity threshold, it may be determined that the gravity distribution is uneven, and a current transmission speed may be reduced by a preset value through the speed control device. If the gravity distribution remains uneven after the transmission speed is reduced, the transmission speed may be further reduced, until the gravity distribution becomes even.

In some embodiments, by detecting the gravity distribution of the medical bed, it is possible to determine whether the medical bed or the subject is unstable. In the event of instability of the medical bed or the subject, the speed of the movable bed board is reduced, which prevents the subject from falling due to fluctuations.

In some embodiments, the medical bed apparatus 2000 may further include an auxiliary support device.

The auxiliary support device may be configured to provide auxiliary support to the movable bed board 2220 on the medical bed board 2120. In some embodiments, the auxiliary support device may be disposed on the medical bed board 2120. In some embodiments, the auxiliary support device may be movable along the length direction (e.g., the Y-axis direction in FIG. 2) of the medical bed board 2120. In some embodiments, at least one side surface of the auxiliary support device may abut against a lower surface of the movable bed board 2220. In some embodiments, the auxiliary support device may move in synchronization with the movable bed board 2220.

In some embodiments, the auxiliary support device may include various structures. For example, the auxiliary support device may include a groove provided along the length direction (e.g., the Y-axis direction in FIG. 2) of the medical bed board 2120, and a slider slidably provided in the groove, where the slider may abut against the movable bed board 2220. As another example, the auxiliary support device may include a screw and a screw nut, where an axis of the screw may be parallel to the length direction (e.g., the Y-axis direction in FIG. 2) of the medical bed board 2120, and the screw nut may be provided on the screw and may abut against the movable bed board 2220.

In some embodiments, the medical bed apparatus 2000 may, in response to determining that an instability is greater than a preset value, enable the auxiliary support device and move the auxiliary support device to a first auxiliary support position. In some embodiments, the first auxiliary support position may be determined based on gravity distribution data, and the instability may be determined based on the gravity distribution data and the transmission speed of the conveying device 2300.

The instability refers to relevant data reflecting a degree of instability of the movable bed board 2220. In some embodiments, the degree of instability may be related to a bending moment of the movable bed board 2220. For example, the greater the bending moment borne by the movable bed board 2220 is, the higher the instability of the movable bed board 2220 is.

In some embodiments, based on the gravity distribution data and the transmission speed, the instability may be determined through a preset table, the preset table including a preset correspondence relationship between the gravity distribution data, the transmission speed, and the instability.

In some embodiments, the preset table may be constructed as follows: for the gravity distribution data and actual transmission speed during a bed plate transfer process of the subject, a count of instances of discomfort feedback from the subject, a count of medical personnel assisting in the transfer, a count of assisted transfers, a sliding frequency and/or a sliding magnitude of the subject relative to the bed plate during the transfer process may be weighted and summed to obtain the instability.

The count of instances of discomfort feedback from the subject refers to a count of times during a transfer process of the movable bed board 2220 that the subject provides feedback of feeling uncomfortable, which may be obtained based on manual statistics.

The count of medical personnel assisting in the transfer refers to a total count of medical personnel assisting in the transfer of the movable bed board 2220 during a transfer process, which may be obtained based on manual statistics.

The count of assisted transfers refers to a count of times the medical personnel participate in assisting in the transfer of the movable bed board 2220 during a transfer process, which may be obtained based on manual statistics.

The sliding frequency of the subject relative to the bed board during the transfer process refers to a frequency at which the subject slides relative to the movable bed board 2220 during a transfer process. In some embodiments, the sliding frequency of the subject relative to the bed plate during the transfer process may be determined based on the gravity distribution data. For example, if a similarity between the gravity distribution data collected at a time point or period (e.g., one minute) before a transfer process and the gravity distribution data collected at a time point or period (e.g., one minute) after the transfer process is less than a preset similarity threshold, it may be determined that the subject has slid. The sliding frequency refers to a count of occurrences of sliding per unit of time.

The sliding magnitude of the subject relative to the bed plate during the transfer process refers to a distance the subject slides relative to the movable bed board 2220 during a transfer process. In some embodiments, the sliding magnitude of the subject relative to the bed plate during the transfer process may be determined based on the gravity distribution data. For example, in a transfer process, a distance between a center of gravity corresponding to the gravity distribution data collected at a time point or period (e.g., one minute) before and a center of gravity corresponding to the gravity distribution data collected at a time point or period (e.g., one minute) later may be designated as the sliding magnitude. An average sliding magnitude may be obtained by taking an average of a plurality of sliding magnitudes.

In some embodiments, based on the count of instances of discomfort feedback from the subject, the count of medical personnel assisting in the transfer, the count of assisted transfers, the sliding frequency of the subject relative to the bed plate during the transfer process, and the average sliding magnitude, the instability may be determined using a preset algorithm (e.g., a weighted algorithm). For example, N=W1×D+W2×M×C+W3×P×S, where W1, W2, and W3 denote preset values, N denotes instability, D denotes the count of instances of discomfort feedback from the subject, M denotes the count of medical personnel assisting in the transfer, C denotes the count of assisted transfers, P denotes the sliding frequency of the subject relative to the bed plate during the transfer process, and S denotes the average sliding magnitude.

The first auxiliary support position is a position where the auxiliary support device needs to support the movable bed board 2220. When the auxiliary support device moves to the first auxiliary support position and supports the movable bed board 2220, the stability of the movable bed board 2220 may be improved.

In some embodiments, the first auxiliary support position may correspond to the location where the bending moment of the movable bed board 2220 is maximum. In some embodiments, based on the gravity distribution data, the gravity magnitude at various positions may be obtained, and by performing force analysis, a position corresponding to a maximum bending moment may be determined, and the position may be determined as the first auxiliary support position.

By way of example, the movable bed board 2220 may be supported from a bottom to a top at two ends of the movable bed board 2220 while the subject lies on the movable bed board 2220. The maximum bending moment is borne at a middle position of the movable bed board 2220, and the first auxiliary support position may be below the middle position of the movable bed board 2220. In other words, the auxiliary support device may support the movable bed board 2220 from below the middle position of the movable bed board 2220. In some embodiments, when the subject lies on the movable bed board 2220, the position on the movable bed board 2220 where the bending moment is maximum may be below the center of gravity of the subject, and the position may be determined as the first auxiliary support position.

In some embodiments, by determining instability, it is possible to assess whether there is a risk of instability for the subject on the movable bed board and accordingly provide targeted auxiliary support to improve the stability of the movable bed board during transfer, thereby preventing the subject from falling off the movable bed board and ensuring the safety of the subject.

In some embodiments, the instability may be determined based on the gravity distribution data and the transmission speed of conveying device 2300 through an instability prediction model.

The instability prediction model refers to a model configured to determine the instability. In some embodiments, the instability prediction model may be a machine learning model, such as a convolutional neural network (CNN) model, etc.

In some embodiments, an input to the instability prediction model may include the gravity distribution data and the transmission speed of the conveying device 2300, and an output of the instability prediction model may include the instability. More descriptions of the gravity distribution data, the transmission speed of the conveying device, and the instability may be found in the relevant descriptions above.

In some embodiments, the instability prediction model may be obtained by training a plurality of first training samples with a first label. The training process may be performed on a remote server. The remote server may input the plurality of first training samples with the first label into an initial instability prediction model, construct a loss function based on the first label and a result of the initial instability prediction model, and iteratively update a parameter of the initial instability prediction model based on the loss function. When the loss function of the initial instability prediction model meets a preset condition, the model training is completed, and the trained instability prediction model is obtained. The preset condition may include the loss function converging or a count of iterations reaching a threshold.

In some embodiments, the first training sample may include the gravity distribution data and the transmission speed of the conveying device obtained from historical data. In some embodiments, in a subsequent subject transfer process, the first label may be obtained by performing a weighted summation on the count of instances of discomfort feedback from the subject, the count of medical personnel assisting in the transfer, the count of assisted transfers, and the sliding frequency and the sliding magnitude of the subject relative to the bed plate during the transfer process in the historical data. More descriptions of the count of instances of discomfort feedback from the subject, the count of medical personnel assisting in the transfer, the count of assisted transfers, the sliding frequency and sliding magnitude of the subject relative to the bed plate during the transfer process, and the weighted summation may be found in the relevant descriptions above.

In some embodiments, predicting the instability through the instability prediction model can improve the accuracy and efficiency of instability prediction.

In some embodiments, as shown in FIG. 2, the transfer bed 2200 may include a lifting seat 2211, with the movable bed board 2220 detachably mounted on the lifting seat 2211. By providing the lifting seat 2211, the lifting seat 2211 may drive the movable bed board 2220 to move up and down (along the Z-axis direction in FIG. 2) to adjust a height of the movable bed board 2220 from a ground, thereby facilitating the transfer of the subject. For example, by adjusting the lifting of the lifting seat 2211 (e.g., lowering the height of the movable bed board 2220), the subject may be transferred to the movable bed board 2220 more easily, and then by adjusting the lifting of the lifting seat 2211 (e.g., raising the height of the movable bed board 2220), the conveying device may dock with the movable bed board 2220 more easily. The lifting seat 2211 may be operable to raise and lower the movable bed board 2220 via the drive device 2320. An exemplary drive device 2320 may include a motor (e.g., a linear motor), a hydraulic cylinder, an air cylinder, etc.

In some embodiments, the lifting seat 2211 may be provided with one or more universal wheels 2212, which may facilitate the movement of the lifting seat in a plurality of directions to facilitate the movement of the transfer bed 2200. In some embodiments, the movable bed body 2210 may include an automatic navigation device. The automatic navigation device may be configured to automatically plan a transfer route of the transfer bed 2200 based on a departure and a destination of the transfer bed 2200, and control the movable bed body 2210 to move along the planned transfer route. By providing the automatic navigation device, the process of transferring the subject with the transfer bed 2200 can be made more intelligent.

In some embodiments, the conveying device 2300 may be detachably docked with the movable bed board 2220 via the docking assembly 2400. The detachable docking between the conveying device 2300 and the movable bed board 2220 may be understood as follows: the conveying device 2300 may transport the movable bed board 2220, but the conveying device 2300 may detach from the movable bed board 2220 after the transmission process ends or after the treatment of the subject is completed. For example, the movable bed board 2220 may be connected to the conveying device 2300 in a detachable manner, or the movable bed board 2220 may be directly placed on the conveying device 2300 (the movable bed board 2220 not connected with the conveying device 2300).

In some embodiments, the docking assembly 2400 may include a first docking member, a second docking member, and a locking mechanism. One of the first docking member and the second docking member may be disposed on the movable bed board 2220, and the other of the first docking member and the second docking member may be disposed on the conveying device 2300. In some embodiments, the first docking member is capable of being docked with the second docking member, and the locking mechanism connects and locks the docked first docking member and the second docking member to stably connect the movable bed board 2220 and the conveying device 2300, thereby facilitating the transmission of the movable bed board 2220 by the conveying device 2300.

In some embodiments, the docking assembly 2400 may further include a detection device 2470 configured to detect whether the first docking member and the second docking member are docked in place. When the first docking member and the second docking member are docked in place, the locking mechanism may be controlled to lock the conveying device 2300 to the movable bed board 2220, allowing the conveying device 2300 to transport the movable bed board 2220.

In some embodiments, the detection device 2470 includes a switch. In some embodiments, when the first docking member and the second docking member are docked in place, the switch is triggered and emits a trigger signal. The locking mechanism may begin the locking operation in response to the trigger signal.

In some embodiments, the detection device 2470 may include a laser sensor. The laser sensor may be configured to detect a relative position and/or a distance between the movable bed board 2220 and the conveying device 2300 (i.e., a relative position and/or a distance between the first docking member and the second docking member) to determine whether the first docking member and the second docking member are docked in place.

FIG. 5 is a schematic diagram illustrating a structure of a docking assembly according to some embodiments of the present disclosure; FIG. 6 is an enlarged schematic diagram at M of FIG. 5; and FIG. 7 is a schematic diagram illustrating a structure of a docking protrusion and a first driving member of the docking assembly according to some embodiments of the present disclosure.

In some embodiments, the docking assembly may include the first docking member, the second docking member, and/or the locking mechanism. In some embodiments, as shown in FIGS. 5-6, the first docking member may include a docking protrusion 2410, and the second docking member may include a docking groove 2420. One of an outer wall of the docking protrusion 2410 and an inner wall of the docking groove 2420 may be provided with a helical groove (not shown in the drawings). It may be understood that the positions of the docking protrusion 2410 and the docking groove 2420 shown in FIGS. 5-6 may be interchanged. In other words, the first docking member may include the docking groove 2420, and the second docking member may include the docking protrusion 2410. In some embodiments, a protrusion part 2411 capable of being inserted into the helical groove may be provided on one of the outer wall of the docking protrusion 2410 and the inner wall of the docking groove 2420. The docking protrusion 2410 is rotatably arranged on the conveying device 2300 or the movable bed board 2220, and the docking protrusion 2410 may be configured to rotate along an axis of the helical groove. When the docking protrusion 2410 and the docking groove 2420 are docked in place, the protrusion part 2411 enters one end of the helical groove. During the rotation of the docking protrusion 2410, the protrusion part 2411 may move along the helical groove so that the docking protrusion 2410 enters the docking groove 2420

FIG. 5 is illustrated as an example where the docking protrusion 2410 is rotatably arranged on the conveying device 2300 (the transfer member 2310), the protrusion part 2411 is disposed on an outer wall of the docking protrusion 2410, and the docking groove 2420 is disposed on the movable bed board 2220, with the helical groove disposed on the inner wall of the docking groove 2420. In some embodiments, a count of the protrusion part 2411 and a count of the helical groove may be more than one (e.g., two, three, etc.), and a plurality of protrusion parts 2411 and a plurality of helical grooves may be arranged in a one-to-one correspondence.

In some embodiments, the locking mechanism may include a first driving member 2450, which may be configured to drive the docking protrusion 2410 to rotate along the axis of the helical groove, causing the protrusion part 2411 to move along the helical groove. By coordinating the docking protrusion 2410 and the docking groove 2420, the docking between the conveying device 2300 and the movable bed board 2220 can be achieved, making the docking between the conveying device 2300 and the movable bed board 2220 stable and reliable.

In some embodiments, the first driving member 2450 may include a motor, a hydraulic cylinder, a gasoline engine, or other drive sources to achieve automatic driving of the docking protrusion 2410. In other embodiments, the first driving member 2450 may be a manual driving component, as shown in FIG. 7. For example, the first driving member 2450 may be a rod or a knob connected to the docking protrusion 2410 (shown as a rod in FIG. 7), and an operator (e.g., a doctor) may manually push the rod or rotate the knob to drive the docking protrusion 2410 to rotate.

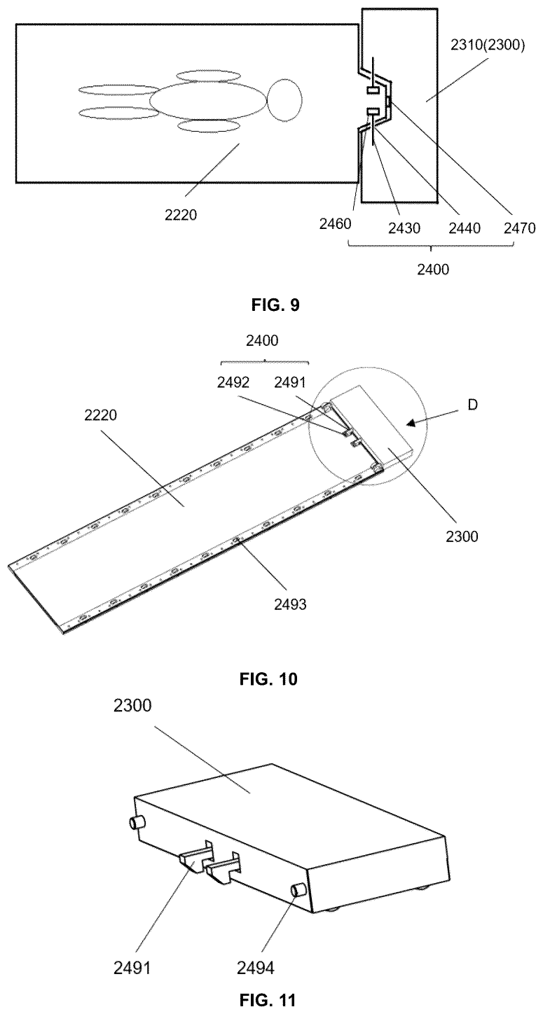

FIG. 8 is a schematic diagram illustrating a structure of the docking assembly according to some other embodiments of the present disclosure; and FIG. 9 is a schematic diagram illustrating a structure of the docking assembly according to some other embodiments of the present disclosure.

In some embodiments, as shown in FIGS. 8-9, the first docking member includes a bolt 2430, the second docking member includes a slot 2440, and the locking mechanism includes a second driving member 2460, which may be configured to drive the bolt 2430 to insert into the slot 2440.

The second driving member 2460 may include a motor (e.g., a linear motor), a hydraulic cylinder, an air cylinder, etc. FIGS. 8-9 illustrate an example where the bolt 2430 is disposed on the movable bed board 2220 and the slot 2440 is disposed on the conveying device 2300. FIG. 8 illustrates a state before the bolt 2430 and the slot 2440 are docked, and FIG. 9 illustrates a state after the bolt 2430 and the slot 2440 are docked. It may be understood that the positions of the bolt 2430 and the slot 2440 may be interchanged, i.e., the bolt 2430 may be disposed on the conveying device 2300, and the slot 2440 may be disposed on the movable bed board 2220.

The movable bed board 2220 may be provided with a housing, and the bolt 2430 and the second driving member 2460 may be located inside the housing. The housing may protect the bolt 2430 and the second driving member 2460. An opening may be provided on the housing at a position where the bolt 2430 extends out. When the movable bed board 2220 and the conveying device 2300 move into position, the second driving member 2460 may drive the bolt 2430 to extend from the opening and then insert into the slot 2440, thereby locking the movable bed board 2220 and the conveying device 2300.

The docking between the conveying device 2300 and the movable bed board 2220 may be achieved by the cooperation of the bolt 2430 and the slot 2440, making the docking between the conveying device 2300 and the movable bed board 2220 stable and reliable.

FIG. 10 is a schematic diagram illustrating a structure of the docking assembly according to yet some more embodiments of the present disclosure; FIG. 11 is a schematic diagram illustrating a structure of a conveying device in the embodiment of FIG. 10; and FIG. 12 is an enlarged schematic diagram of part D of FIG. 10. In some embodiments, see FIGS. 10-12, the first docking member of the docking assembly 2400 may include a clasp 2491 and the second docking member includes a clasp slot 2492. The locking mechanism includes a third driving member, which may be configured to drive the clasp 2491 to hook into pr withdraw from the clasp slot 2492. For example, the third driving member may be configured to drive the clasp along a depth direction of the clasp slot 2492, allowing the clasp 2491 to hook into or withdraw from the clasp slot 2492. As another example, the third driving member may also drive the clasp 2491 to rotate such that the clasp 2491 may hook into the clasp slot 2492 or exit from the clasp slot 2492. The third driving member may be a motor, a hydraulic cylinder, or the like.

In some embodiments, a locking device 2493 (e.g., a cam), a fourth driving member configured to drive the movement of the locking device 2493 (e.g., drives rotation of the cam), and a triggering mechanism 2494 may be provided on the movable bed board 2220. The fourth driving member may be configured to drive the locking device 2493 to move, allowing the movable bed board 2220 to move in the length direction of the movable bed board 2220. The triggering mechanism 2494 may be provided on the conveying device 2300, and when the movable bed board 2220 approaches the conveying device 2300, the triggering mechanism 2494 may be triggered. Then, the fourth driving member may drive the locking device 2493 to move (e.g., driving the cam to rotate), causing the movable bed board 2220 to move a short distance in a direction away from the conveying device 2300, thereby ensuring that the clasp 2491 is securely engaged in the clasp slot 2492. In other words, when the clasp 2491 is hooked into the clasp slot 2492, a gap may exist between the clasp 2491 and the clasp slot 2492. By driving the movable bed board 2220 to move a short distance away from the conveying device 2300, the locking device 2493 eliminates the gap between the clasp 2491 and the clasp slot 2492, ensuring that the clasp 2491 is securely hooked into the clasp slot 2492.

In some other embodiments, the first docking member, the second docking member, and the locking mechanism may be of other structures.

In some embodiments, the first docking member may further include a first actuating member, and the second docking member may further include a second actuating member. In some embodiments, the first actuating member and the second actuating member may be configured to connect and disconnect the first docking member and the second docking member.

In some embodiments, the first actuating member and the second actuating member may be configured to generate an interacting force to connect or disconnect the first docking member and the second docking member. In some embodiments, the interacting force may be a magnetic force. At least one of the first actuating member, the second actuating member may generate a magnetic force and/or be magnetically attracted. For example, one of the first actuating member and the second actuating member may be a magnet, and the other of the first actuating member and the second actuating member may be a magnet or a metal that may be magnetically attracted, such as iron, nickel, or the like. In some embodiments, the magnet may include a permanent magnet and/or an electromagnet.

Merely by way of example, the first docking member may include an electromagnet, the second docking member may be a magnetic component, and the locking mechanism may be an electrically connected switch associated with the electromagnet. When the movable bed board 2220 and the conveying device 2300 move into position, the electrically connected switch connected to the electromagnet may be turned on, causing the electromagnet to be energized and attract the magnetic component, thereby connecting and locking the movable bed board 2220 and the conveying device 2300. By changing a magnitude of a current, the magnetic force may be further adjusted to change the degree of connection.

In some embodiments, the docking assembly 2400 disposed on the medical bed 2100 further includes an anti-skid device, which is configured to prevent the docking assembly 2400 on the medical bed board 2120 from sliding.

In some embodiments, the anti-skid device may include one or more anti-skid strips or one or more anti-skid platforms disposed on at least one of the first docking member and the second docking member. In some embodiments, one of the first docking member and the second docking member may be slidably connected to the anti-skid platform disposed on the other one of the first docking member and the second docking member. By providing the one or more anti-skid strips or the one or more anti-skid platforms, the friction between the first docking member and the second docking member can be increased to prevent slippage.

In some embodiments, an anti-skid pattern may be set on the one or more anti-skid platforms. By providing the anti-skid pattern, the friction between the first docking member and the second docking member can be further increased.

By utilizing the advantages of a simple structure and convenient control provided by the interaction force to connect the first docking member and the second docking member, the connection structure can be simplified. By providing the anti-skid device, the friction between the first docking member and the second docking member can be increased, thereby improving the connection effectiveness and preventing slippage.

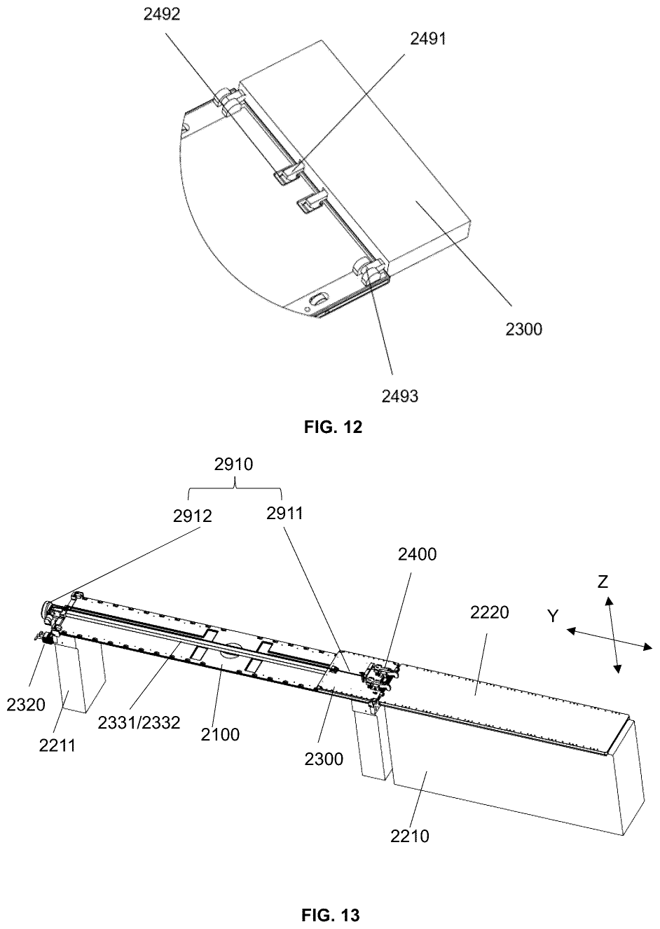

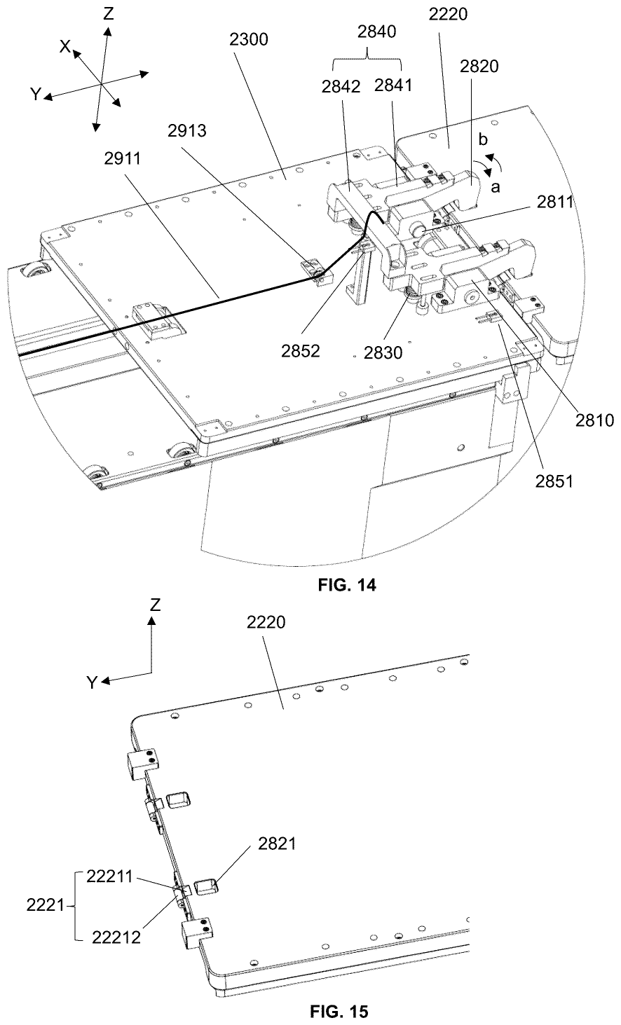

FIG. 13 is an exemplary schematic diagram illustrating a structure of the medical bed apparatus according to some embodiments of the present disclosure; FIG. 14 is an exemplary schematic diagram of a hook connected with a docking groove according to some embodiments of the present disclosure; and FIG. 15 is a schematic diagram illustrating a localized structure of a movable bed board according to some embodiments of the present disclosure.

In some embodiments, as shown in FIGS. 13-15, the first docking member includes at least one hook 2820, the second docking member includes at least one docking groove 2821, the at least one hook 2820 is provided on one of the conveying device 2300 and the movable bed board 2220, and the at least one docking groove 2821 is provided on another one of the conveying device 2300 and the movable bed board 2220. The at least one hook 2820 is capable of being connected with or disconnected from the docking groove 2821 by rotation.

In some embodiments, the docking assembly 2400 includes a bracket 2810, which may be provided on the conveying device 2300. In some embodiments, the bracket 2810 may also be disposed on the movable bed board 2220, and the docking groove 2821 is disposed on the conveying device 2300.

The hooks 2820 are configured to attach the docking assembly 2400 to the movable bed board 2220.

A count of hooks 2820 may be set according to actual needs. Exemplarily, as shown in FIG. 14, the docking assembly 2400 includes two hooks 2820.

In some embodiments, the docking assembly 2400 further includes a bracket 2810, and the bracket 2810 is configured to support the hooks 2820. In some embodiments, the bracket 2810 is secured to the conveying device 2300 when the hook 2820 is rotatably disposed on the conveying device 2300, as shown in FIG. 14. For example, the bracket 2810 may be secured to the conveying device 2300 by a lock nut. As another example, the bracket 2810 and the conveying device 2300 may be integrally molded. In some embodiments, the bracket 2810 is fixed to the movable bed board 2220 when the hook 2820 is rotatably disposed to the movable bed board 2220.

In some embodiments, the hook 2820 may include a connecting rod and a hook-shaped portion, and the connecting rod is rotatably connected to the bracket 2810. The hook-shaped portion is a curved hook structure, and the hook-shaped portion cooperates with the docking groove 2821 to remain stably connected to the docking groove 2821.

The docking groove 2821 refers to a groove provided in the movable bed board 2220. In some embodiments, a count of docking grooves 2821 is the same as the count of hooks 2820. The docking grooves 2821 correspond one-to-one with the hooks 2820. Exemplarily, as shown in FIG. 14, when the docking assembly 2400 includes two hooks 2820 and two docking grooves 2821, the two hooks 2820 and the two docking grooves 2821 are in one-to-one correspondence. In some other embodiments, the count of docking grooves 2821 may be more than the count of hooks 2820.

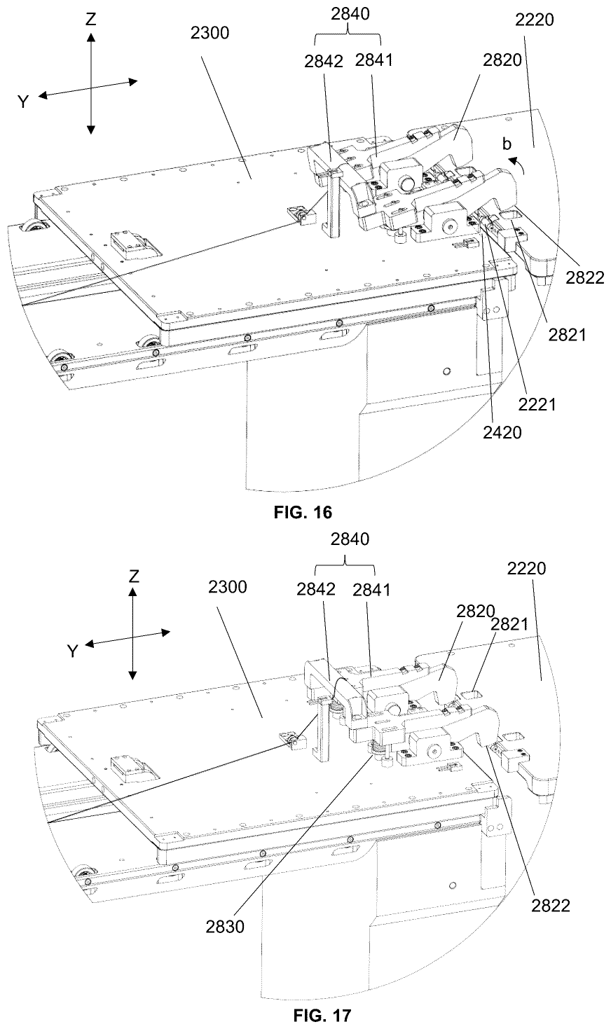

FIG. 16 is an exemplary schematic diagram illustrating a structure of the hook disconnected from the docking groove according to some embodiments of the present disclosure; and FIG. 17 is an exemplary schematic diagram illustrating a structure of the movable bed board disconnected from the conveying device according to some embodiments of the present disclosure. The following is illustrated in conjunction with FIGS. 14 and 16-17.

In some embodiments, by rotating the hook 2820, the hook 2820 can connect with or disconnect from the docking groove 2821, allowing the conveying device 2300 to connect with or disconnect from the movable bed board 2220. Prior to the commencement of an operation, as shown in FIG. 14, the subject is positioned on the movable bed board 2220, and after the conveying device 2300 is moved into place (e.g., the movable bed board 2220 is detected to have arrived at a position at which the docking assembly 2400 is capable of realizing connecting by a first sensor 2851 described below), the hook 2820 is rotated to lift up (e.g., the hook 2820 is rotated along a direction of direction b in FIG. 14), and the hook 2820 is rotated to fall (e.g., the hook 2820 is rotated along a direction of direction a in FIG. 14) to connect with the docking groove 2821, so that the conveying device 2300 realizes connecting with the movable bed board 2220, enabling the conveying device 2300 to drive the movable bed board 2220 to move to the medical bed 2100, thereby transferring the subject to the medical bed 2100. After the operation is completed, as shown in FIG. 16, the conveying device 2300 is moved to drive the movable bed board 2220 away from the medical bed 2100, the hook 2820 is rotated to lift up (e.g., the hook 2820 is rotated in the direction b in FIGS. 14 and 16) until it is disconnected from the docking groove 2821, and the movable bed board 2220 is controlled to move away from the conveying device 2300 (by manually dragging it or transferring through the movable bed body 2210) to disconnect the movable bed board 2220 from the conveying device 2300 (as shown in FIG. 17).

The design of the docking assembly 2400 ensures a firm connection between the movable bed board 2220 and the conveying device 2300, thereby ensuring the smoothness and safety of the entire transfer process. By setting the at least one hook 2820, the rapid connecting and disconnecting of the movable bed board 2220 and the conveying device 2300 can be realized, thereby improving the transfer efficiency.

In some embodiments, when the movable bed board 2220 is on the movable bed body 2210, the relative position between the conveying device 2300 and the movable bed board 2220 may also be changed by lowering the movable bed body 2210 or raising the medical bed 2100 so that the hook 2820 disengages from the docking groove 2821. When the movable bed board 2220 moves to a position close to the conveying device 2300, the height of the medical bed 2100 may be raised or the height of the movable bed body 2210 may be lowered so that the movable bed board 2220 is located below the hook 2820, and the position of the movable bed body 2210 may be adjusted so that a projection of the hook 2820 coincides with the docking groove 2821. Then, lowering the height of the medical bed 2100 or raising the movable bed body 2210 allows the hook 2820 to engage with the docking groove 2821.

In some embodiments, a resilient layer is provided within the docking groove 2821. The resilient layer refers to a structure for enhancing docking stability. When the hook 2820 is not connected to the docking groove 2821, the resilient layer is not squeezed, and when the hook 2820 is connected to the docking groove 2821, the resilient layer is squeezed, generating an elastic deformation and exerting a certain pressure on the hook 2820, which increases the friction between the hook 2820 and the docking groove 2821, thereby enhancing docking stability. Exemplarily, the resilient layer may be a silicone layer, a rubber layer, or the like.

In some embodiments, the position of the docking groove 2821 is capable of being adjustable relative to the movable bed board 2220, i.e., the docking groove 2821 is capable of moving in the length direction or a width direction of the movable bed board 2220. For example, a docking rail can be provided on the movable bed board 2220, and the docking groove 2821 is provided on the docking rail so that the docking groove 2821 is capable of sliding on the docking rail relative to the movable bed board 2220, and when the sliding to a position corresponding to the hook 2820, the position of the docking groove 2821 is locked by a locking structure provided on the docking groove 2821 or other feasible means. The docking rail may be provided along the length direction (e.g., the Y-axis direction) and/or the width direction (e.g., the X-axis direction) of the movable bed board 2220.

When connecting is performed, the position of the movable bed board 2220 may sometimes be not quite appropriate, for example, the movable bed board 2220 is far away from the hooks 2820 or shifted from side to side, and by setting the position of the docking groove 2821 to be able to adjust relative to the movable bed board 2220, connecting can be ensured in various scenarios.

In some embodiments, the docking groove 2821 may also be provided at the bottom or side of the movable bed board 2220, and accordingly, based on the position of the docking groove 2821, the hook 2820 may be provided at a corresponding position of the conveying device 2300 with a set in a corresponding direction of rotation. For example, when the docking groove 2821 is provided at the bottom of the movable bed board 2220, the hook 2820 is also provided at the bottom of the conveying device 2300, and the hook 2820 is raised upwardly when connecting is performed, and the hook 2820 falls down when disconnecting is performed. For example, when the docking grooves 2821 are provided on both sides of the movable bed board 2220, the hooks 2820 are also provided on both sides of the conveying device 2300, and the hooks 2820 are rotated in a direction close to the center axis of the movable bed board 2220 when connected, and the hooks 2820 are rotated in a direction away from the center axis of the movable bed board 2220 when disconnected. The above settings can enable the medical bed apparatus 2000 to be applied in more scenarios, and it may be set according to the actual needs.

In some embodiments, the structure of the docking groove 2821 is not limited to a groove opened on the movable bed board 2220, but may also be a hook-shaped structure, and an inner side of the hook-shaped structure may be a groove that matches the hook 2820.

In some embodiments, as shown in FIGS. 14 and 16-17, the docking assembly 2400 further includes at least one resilient member 2830 and an unlocking member 2840, the at least one hook 2820 is disposed on the unlocking member 2840, and the at least one resilient member 2830 is arranged between the conveying device 2300 and the unlocking member 2830 The resilient member 2830 is configured to provide a resilient return force to the hook 2820. The resilient member 2830 may be a coil spring, a torsion spring, a rubber pad, etc. The at least one resilient member 2830 keeps the at least one hook 2820 connected to the at least one docking groove 2821 under a resilient return force. In some embodiments, when the at least one hook 2820 is connected to the at least one docking groove 2821, the at least one resilient member 2830 is in a state of resilient deformation to provide a resilient return force to keep the at least one hook 2820 and the at least one docking groove 2821 docked.

The unlocking member 2840 is configured to control the rotational state of the hook 2820. In some embodiments, the unlocking member 2840 may be controlled manually or automatically such that the unlocking member 2840 drives the at least one hook 2820 to rotate relative to the bracket 2810. In some embodiments, the unlocking member 2840 includes a release handle 2841 and at least one pressure plate 2842. The at least one hook 2820 can be easily controlled to rotate relative to the bracket 2810 by controlling the release handle 2841. The at least one pressure plate 2842 is configured to connect the release handle 2841 and the at least one hook 2820. In some embodiments, the at least one pressure plate 2842 is provided above the at least one hook 2820, and the release handle 2841 is removably connected to the at least one pressure plate 2842. The resilient member 2830 is provided between the platen 2842 and the conveying device 2300.

In some embodiments, the release handle 2841 may be pushed or pulled by a manual or the drive mechanism 2910, which may cause the at least one pressure plate 2842 to drive the at least one hook 2820 to rotate relative to the bracket 2810. In some embodiments, a count of the release handle 2841 may be one, and the at least one pressure plate 2842 may have a one-to-one correspondence with the at least one hook 2820. By pushing or pulling the release handle 2841, the at least one pressure plate 2842 can be driven, which in turn drives the at least one hook 2820 to rotate.

In some embodiments, the bracket 2810 is provided with a hook rotating shaft 2811. The hook 2820 may rotate around the hook rotating shaft 2811. In some embodiments, as shown in FIG. 14, for example, the docking assembly 2400 includes two hooks 2820, and both of the two hooks 2820 are rotatably disposed on the hook rotating shaft 2811.

In some embodiments, the hooks 2820 and the resilient member 2830 are disposed on each side of the hook rotating shaft 2811 along the conveying direction of the conveying device 2300. When the hook 2820 is lifted or is in the connecting state, the resilient member 2830 is compressed and deformed to generate a resilient return force, and the hook 2820 automatically falls down under the action of the resilient return force and stays in the state of connecting with the docking groove 2821. The conveying direction of the conveying device 2300 refers to a direction of movement of the conveying device 2300 when transferring the movable bed board 2220. Exemplarily, the conveying direction may be the Y-axis direction, as shown in FIGS. 13-14.

In some other embodiments, the hook 2820 and the hook rotating shaft 2811 are located on both sides of the resilient member 2830 along the conveying direction of the conveying device 2300 (e.g., the Y-axis direction in FIGS. 13-14), and the resilient member 2830 is stretched and deformed to generate a resilient return force when the hook 2820 is lifted up or in the docking state, so that the hook 2820 is able to fall down under an action of the resilient return force automatically and maintain connecting with the docking groove 2821.

In some embodiments of the present disclosure, by providing the resilient member 2830 and the unlocking member 2840, it is possible to enable the hook 2820 to realize an automatic reset to be docked with the docking groove 2821, and to make the hook 2820 and the docking groove 2821 always keep a close connecting, avoiding detachment due to external force or vibration, thereby realizing the effect of stabilization, automatic resetting, and convenient releasing.

In some embodiments, the conveying device 2300 and the movable bed board 2220 may be manually connected or disconnected. In the embodiment shown in FIG. 14, for example, prior to the start of the operation, when the conveying device 2300 is moved into place near the movable bed board 2220, the release handle 2841 is manually pressed to cause the hook 2820 to lift up, the release handle 2841 is released, and the hook 2820 falls under the action of gravity or the action of the resilient return force of the resilient member 2830 and connects with the docking groove 2821. The hook 2820 remains more stably connected with the docking groove 2821 under the resilient return force of the resilient member 2830. As another example, after the operation is completed, the conveying device 2300 may convey the movable bed board 2220 to one end of the medical bed 2100, and the release handle 2841 is manually pressed to cause the hook 2820 to lift up and the hook 2820 disconnects from the docking groove 2821.

In some embodiments, as shown in FIGS. 13-14, the medical bed apparatus further includes a drive mechanism 2910 driving the at least one hook 2820 to rotate, the drive mechanism 2910 includes a drive cable 2911 and a reel 2912. The reel 2912 is disposed at one end of the medical bed 2100 that is far from a docking location of the conveying device 2300 and the movable bed board 2220, and the drive cable 2911 connects the reel 2912 and the unlocking member 2840.

The drive mechanism 2910 is configured to drive the hook 2820 to rotate so as to enable connecting or disconnecting of the hook 2820 with the docking groove 2821.

The drive cable 2911 is configured to transmit the power generated by the rotation of the reel 2912 to the unlocking member 2840. In some embodiments, the drive cable 2911 is made of a high-strength, abrasion-resistant material, such as a steel wire rope, a synthetic fiber rope, etc.

The reel 2912 is configured to provide power by rotating and thereby winding or releasing the drive cable 2911. The reel 2912 may be driven by a motor or a manual crank. The drive cable 2911 may be wound on the reel 2912.

In some embodiments, one end of the drive cable 2911 is secured to the reel 2912 and the other end is connected to the unlocking member 2840. When the reel 2912 is rotated, the drive cable 2911 is rewound or released, driving the hook 2820 through the unlocking member 2840.

In some embodiments of the present disclosure, by setting the drive cable 2911 and the reel 2912, an automated control of the rotation of the hook 2820 is realized, which reduces the manual operation, and improves the operation efficiency and convenience of the device. The reel is provided at an end of the medical bed away from the docking location of the conveying device 2300 and the movable bed board 2220, which reasonably utilizes the spatial layout of the medical bed 2100, avoids interfering with the other functional components, and improves the overall compactness and beauty of the device.

In some embodiments, one end of the drive cable 2911 is fixed to the reel 2912, while the other end is connected to the unlocking member 2840. When the reel 2912 rotates, the drive cable 2911 is either wound up or released, driving the hook 2820 to rotate through the unlocking member 2840.

In some embodiments, as shown in FIG. 14, the drive mechanism 2910 further includes an alignment guide mechanism 2913, the alignment guide mechanism 2913 is disposed on the conveying device 2300, and the alignment guide mechanism 2913 guides the drive cable 2911.

The alignment guide mechanism 2913 is configured to guide the drive cable 2911 and restrict the movement of the drive cable 2911, so as to avoid that the drive cable 2911 affects the operation of the medical bed apparatus due to bending or deviation. In some embodiments, the alignment guide mechanism 2913 may be a roller or a ring structure with an annular groove. The drive cable 2911 may be wound around a roller or within an annular groove of the annular structure.

In some embodiments of the present disclosure, in some cases, a distance between the reel 2912 and the hook 2820 may be far away, and by providing the alignment guide mechanism 2913, it can be ensured that the drive cable 2911 can be stabilized in all cases to bring the power generated by the rotation of the reel 2912 to the unlocking member 2840.

In some embodiments, the conveying device 2300 and the movable bed board 2220 may be actively connected or disconnected via the docking assembly 2400. For example, prior to an operation, the conveying device 2300 moves toward the movable bed board 2220, and the reel 2912 releases a corresponding length of wire with the movement of the conveying device 2300. After the conveying device 2300 has moved into place (as detected by the first sensor 2851 described below that the movable bed board 2220 has reached a position where the docking assembly 2400 is capable of docking), the reel 2912 rewinds the drive cable 2911 thereby pulling on the release handle 2841, causing the hook 52 to lift up, continuing to control the conveying device 2300 in close proximity to the movable bed board 2220, the reel 2912 releases the drive cable 2911 thereby controlling the hook 2820 to fall, and the hook 2820 docks with the docking groove 2821 on the movable bed board 2220. As another example, after the operation is completed, the conveying device 2300 may transfer the movable bed board 2220 to the end of the medical bed 2100, and the reel 2912 winds up the drive cable 2911 thereby pulling the release handle 2841, causing the hook 52 to lift and the hook 2820 to disconnect from the docking groove 2821.

In some embodiments, as shown in FIGS. 15, 16 and 17, a first guiding slope 2822 is provided on each of at least onehook 2820, and a guiding member 2221 is provided on the movable bed board 2220, with the guide member 2221 and the at least one docking groove 2821 arranged along a conveying direction of the conveying device 2300 conveying direction. The guiding member 2221 cooperates with the first guiding slope 2822.

The first guiding slope 2822 is an inclined surface disposed on a lower portion of the hook 2820 near an end of the movable bed board 2220. There is an acute or obtuse angle between the first guiding slope 2822 and the medical bed board of the medical bed 2100 or the movable bed board 2220.

The guiding member 2221 refers to a component that guides the hook 2820 into the docking groove 2821 by cooperating with the first guiding slope 2822, which in turn is configured to guide the rotation of the hook 2820 during a movement of the conveying device 2300 towards the movable bed board 2220.

In some embodiments, the first guiding slope 2822 is provided in one-to-one correspondence with the guiding member 2221, and the guiding member 2221 is provided in one-to-one correspondence with the hook 2820.

Through the contact between the first guiding slope 2822 and the guiding member 2221, the hook 2820 may be guided to move so as to ensure that the docking groove 2821 can be correctly aligned with, or snapped into, the hook 2820 as it moves. Another option for realizing the lifting of the hook 2820 ensures that the hook 2820 can be lifted in a stable manner.

In some embodiments, the guiding member 2221 includes a second guiding slope 22211. The second guiding slope 22211 refers to a beveled surface on the movable bed board 2220 for guiding movement of the hook 2820. The second guiding slope 22211 may at least partially abut the guiding slope 2822, and the second guiding slope 22211 has an inclined direction and an inclined angle consistent with the guiding slope 2822.

In some embodiments, the guiding member 2221 includes a guiding wheel 22212. The guiding wheel 22212 refers to a rolling member disposed at an edge of the movable bed board 2220. When the movable bed board 2220 is near the conveying device 2300, the guiding wheel 22212 comes into contact with the guiding slope 2822 of the hook 2820, and the guiding wheel 22212 rolls, driving the hook 2820 to rotation, and guides the direction of movement of the guiding slope 2822.

In some embodiments, the guiding member 2221 includes the second guiding slope 22211 and the guiding wheel 22212, and the second guiding slope 22211 and the guiding wheel 22212 is lined up in the conveying direction of the conveying device 2300. Exemplarily, the second guiding slope 22211 and the guiding wheel 22212 are arrayed along a Y-axis direction, as shown in FIG. 15. The guiding wheel 22212 can avoid the hook 2820 from being stuck or deflected during the movement of the hook 2820, and the second guiding slope 22211 can help the hook 2820 to adjust its position before it enters the docking groove 2821, thereby improving the accuracy of guiding. By the cooperation of the second guiding slope 22211 and the guiding wheel 22212, it can better guide the hook 2820 to connect with the docking groove 2821 to achieve more efficient, stable, and reliable transferring.

In some embodiments of the present disclosure, by cooperating the guiding wheel 22212 and/or the second guiding slope 22211 with the guiding slope 2822 on the hooks, the rotation or adjustment of the hook 2820 can be guided to achieve precise docking with the movable bed board 2220. Meanwhile, the design of the guiding wheels 22212 can reduce friction between the hook 2820 and the movable bed board 2220, extending the life of the device.

In some embodiments, the conveying device 2300 and the movable bed board 2220 may be passively realized connected or disconnected via the docking assembly 2400. In the embodiment shown in FIG. 16, for example, prior to the start of an operation, during the movement of the conveying device 2300 toward the movable bed board 2220, the guiding slope 2822 of the hook 2820 is in contact with the guiding wheel 22212 at the edge of the movable bed board 2220, which forces the hook 2820 to lift up; at the same time, the conveying device 2300 continues to approach the movable bed board 2220, and the hook 2820 slides along the second guiding slope 22211 on the movable bed board 2220 until it reaches the docking groove 2821; and the hook 2820 stays connecting with the docking groove 2821 under the force of gravity and the resilient return force of the resilient member 2830. For example, at the end of the surgery, when the movable bed board 2220 is completely disconnected from the medical bed 2100, a height of the medical bed 2100 is controlled to be raised or the height of the movable bed body 2210 is controlled to be lowered, so that the hook 2820 is disconnecting from the docking groove 2821.

In some embodiments, as shown in FIG. 14, the docking assembly 2400 includes a first sensor 2851, and the first sensor 2851 is configured to detect whether the movable bed board 2220 and the conveying device 2300 reach a docking assembly 2400 a position where docking can be achieved. In some embodiments, as shown in FIG. 14, the docking assembly 2400 includes a second sensor 2852, and the second sensor 2852 is configured to detect whether a state of the hook 2820 is in a state that the hook 2820 is connected with the docking groove 2821.

In some embodiments, the first sensor 2851 and the second sensor 2852 may be fiber optic sensors. The fiber optic sensors are not susceptible to environmental influences when used in specific medical scenarios (e.g., radiation therapy scenarios, etc.).

In some embodiments, the first sensor 2851 includes an emitting component and a receiving component. The emitting component of the first sensor 2851 may emit light (e.g., a laser). The receiving component may receive light reflected back. The first sensor 2851 may determine, based on the reception of light by the receiving component (e.g., whether or not reflected light is received, the intensity of the reflected light, etc.), whether or not the movable bed board 2220 and the conveying device 2300 have arrived at a position where the docking assembly 2400 is capable of achieving docking. The second sensor 2852 is similar to the first sensor 2851. In some embodiments, the second sensor 2852 may determine that the hook 2820 is connected with the docking groove 2821 based on the receipt by the receiving component of the reflected light emitted by the emitting component.

In some embodiments, the first sensor 2851 and the second sensor 2852 may also be an infrared sensor, a contact switch, or the like.

In some embodiments, when the first sensor 2851 detects that the movable bed board 2220 and the conveying device 2300 have reached a position where the docking assembly 2400 is capable of realizing docking, the second sensor 2852 detects that the state of the hook 2820 is the state when it is connected to the docking groove 2821 (i.e., the hook 2820 is already in the state of falling down), and then controls the conveying device 2300 to convey the movable bed board 2220 for safety.

In some embodiments of the present disclosure, the first sensor 2851 and the second sensor 2852 can detect and provide feedback on the position of the movable bed board 2220 and the state of the hook 2820, thereby improving the accuracy of the docking and ensuring the safety of the subject.

In some embodiments, as shown in FIG. 13, the medical bed apparatus 2000 further includes a belt drive mechanism 2331 disposed on the medical bed 2100, and the conveying device 2300 moves on the medical bed 2100 through the belt drive mechanism 2331, thereby realizing the transferring of the movable bed board 2220. The drive belt of the belt drive mechanism 2331 moves synchronously with the drive cable 2911.

The drive belt of the belt drive mechanism 2331 is provided along the length of the medical bed 2100 (e.g., in the Y-axis direction in FIG. 13). The belt drive mechanism 2331 may transmit power through the friction of the drive belt to drive the conveying device 2300 to move along the drive belt, which in turn transfers the movable bed board 2220 to the medical bed 2100.

In some other embodiments, the transmission assembly further includes a chain drive mechanism 2332 disposed on the medical bed 2100, and the conveying device 2300 is moved on the medical bed 2100 by the chain drive mechanism 2332, thereby realizing the transferring of the movable bed board 2220. The drive chain of the chain drive mechanism 2332 moves synchronously with the drive cable 2911.

The drive chain of the chain drive mechanism 2332 is provided along the length direction of the medical bed 2100 (e.g., the Y-axis direction in FIG. 13). The chain drive mechanism 2332 may transmit power through the rigid connection between the drive chain and the sprocket to drive the conveying device 2300 to move along the drive belt, which in turn transfers the movable bed board 2220 to the medical bed 2100.

When the drive belt or the drive chain drives the conveying device 2300 to move, the drive cable 2911 moves in synchronization with the drive belt or the drive chain.

In some embodiments of the present disclosure, by setting up the belt drive mechanism 2331 or the chain drive mechanism 2332, the synchronized movement of the drive belt or the drive chain with the drive cable 2911 can ensure that the drive cable 2911 dose not bend or slip out of position during the process of transferring the movable bed board 2220 by the conveying device 2300, and the drive cable 2911 will not accidentally actuate the unlocking member 2840 to cause the hook 2820 to disconnect from the docking groove 2821.

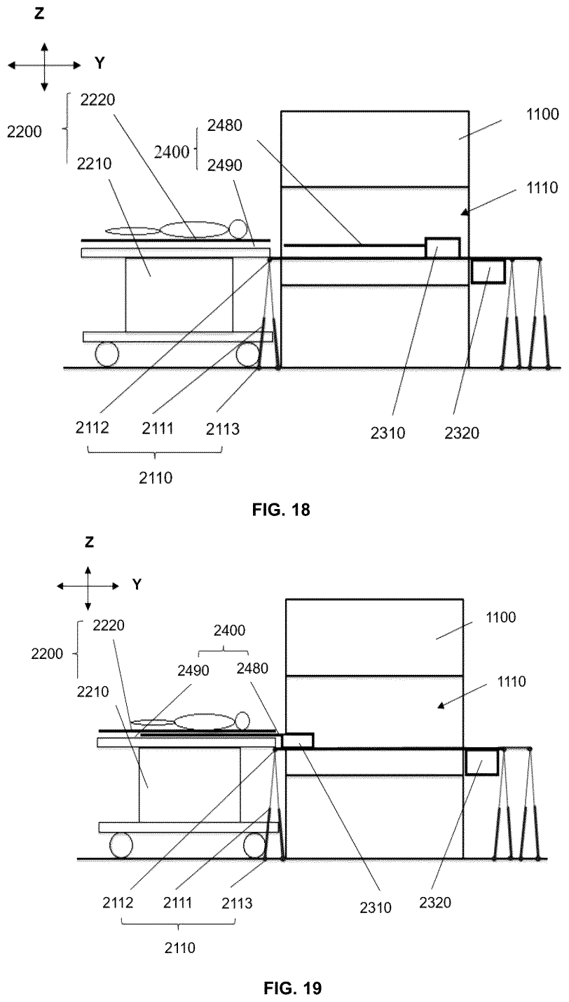

FIG. 18 is a schematic diagram illustrating a structure of the docking assembly according to some further embodiments of the present disclosure.

In some embodiments, as shown in FIG. 18, the docking assembly 2400 may include an operating member 2860, a locking tongue (not shown in the drawings), and a locking hole (not shown in the drawings), with the operating member 2860 and the locking tongue provided on the conveying device 2300, and the locking hole provided on the movable bed board 2220. The operating member 2860 is connected to the locking tongue and may actuate the locking tongue to extend or retract to achieve connecting or disconnecting with the locking hole. In some embodiments, the operating member 2860 and the latch may be coupled by an elastic member 2870. In some embodiments, the operating member 2860 may be an operating knob or may be an operating pull. In some embodiments, the elastic member 2870 may be a torsion spring or a spring.

The locking tongue and the locking hole are configured to cooperate to effectuate disengagement and docking of the conveying device 2300 with the movable bed board 2220. In some embodiments, a bevel is provided on the latch and a locking block (not shown in the drawings) is provided on the movable bed board 2220. The locking block cooperates with the beveled surface of the locking tongue, so that during movement of the conveying device 2300 toward the movable bed board 2220, the cooperation of the bevel and the locking block can push the locking tongue to retract, and the elastic member 2870 undergoes an elastic deformation. Under the action of the elastic recovery force of the elastic member 2870, when the lock tongue moves to the position of the lock hole, the lock tongue pops out, thereby realizing the docking of the conveying device 2300 and the movable bed board 2220. When it is necessary to disconnect the conveying device 2300 from the movable bed board 2220, the operating member 2860 may be operated to drive the lock tongue to remain retracted while moving the movable bed board 2220, and then the operating member 2860 may be released to realize the disengagement of the conveying device 2300 from the movable bed board 2220.

In some embodiments of the present disclosure, by providing the docking assembly including the operating member, the locking tongue, and the locking hole, a quick docking of the movable bed board and the conveying device can be realized, which helps to improve the efficiency of the diagnosis and treatment.

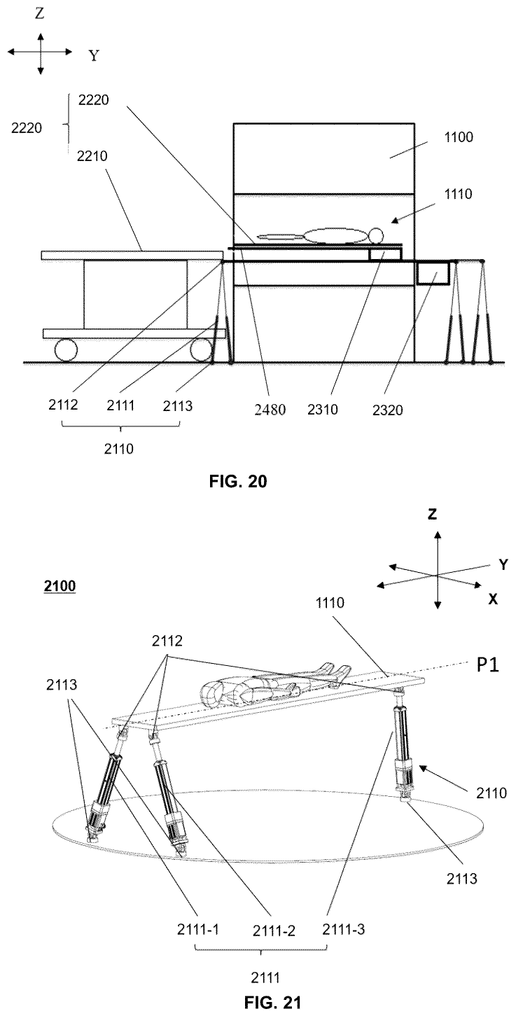

FIG. 19 is a schematic diagram illustrating a structure of the docking assembly according to some further embodiments of the present disclosure; FIG. 20 is a schematic diagram illustrating a structure of the docking assembly according to yet other embodiments of the present disclosure; and FIG. 21 is a schematic diagram illustrating a structure of the docking assembly according to yet other embodiments of the present disclosure.

In some embodiments, see FIGS. 19-21, the docking assembly 2400 may include a receiving plate 2480 and a gap 2490, the receiving plate 2480 is disposed on the conveying device 2300, the gap 2490 is disposed between the movable bed body 2210 and the movable bed board 2220, and the receiving plate 2480 may be configured to be inserted into the gap 2490 to support the movable bed board 2220.

FIGS. 19-21 illustrate an example where the medical bed apparatus 2000 is applied in a medical system. FIG. 19 illustrates a state when the receiving plate 2480 is not inserted into the gap 2490 (the movable bed board 2220 is disconnected with the conveying device 2300). FIG. 20 illustrates a state after the receiving plate 2480 is inserted into the gap 2490 (the movable bed board 2220 is connected with the conveying device 2300). FIG. 21 illustrates a state after the receiving plate 2480 moves the movable bed board 2220 onto the medical bed 2100.

In some embodiments, after the receiving plate 2480 is inserted into the movable bed board 2220, the receiving plate 2480 may lift the movable bed board 2220 upward to detach the movable bed board 2220 from the movable bed body 2210. In some embodiments, when the movable bed body 2210 includes a lifting seat 2211, after the receiving plate 2480 is inserted into the movable bed board 2220, the lifting seat 2211 may descend to detach the movable bed board 2220 from the movable bed body 2210, and the movable bed board 2220 may be supported by the receiving plate 2480.

In some embodiments, a plurality of support rods may be provided between a bottom of the movable bed board 2220 and the movable bed body 2210, creating the gap 2490 between the movable bed board 2220 and the movable bed body 2210.

In some embodiments, when the conveying device 2300 has a transfer member 2310, a receiving plate 2480 is provided on a side of the transfer member 2310 near the movable bed board 2220. By the cooperation of the receiving plate 2480 with the gap 2490, docking of the conveying device 2300 to the movable bed board 2220 is achieved, which makes the structure of the docking assembly 2400 simple and easy to operate.

In some embodiments, the support device 2110 may include at least two robotic arms (not shown in the drawings), and the at least two robotic arms may be disposed at each end of the medical bed board 2120 of the medical bed 2100. The robotic arm may include a plurality of joints articulated in sequence, and by the setup of the robotic arm, it is possible to make the medical bed 2100 have a plurality of degrees of freedom, such as six degrees of freedom.

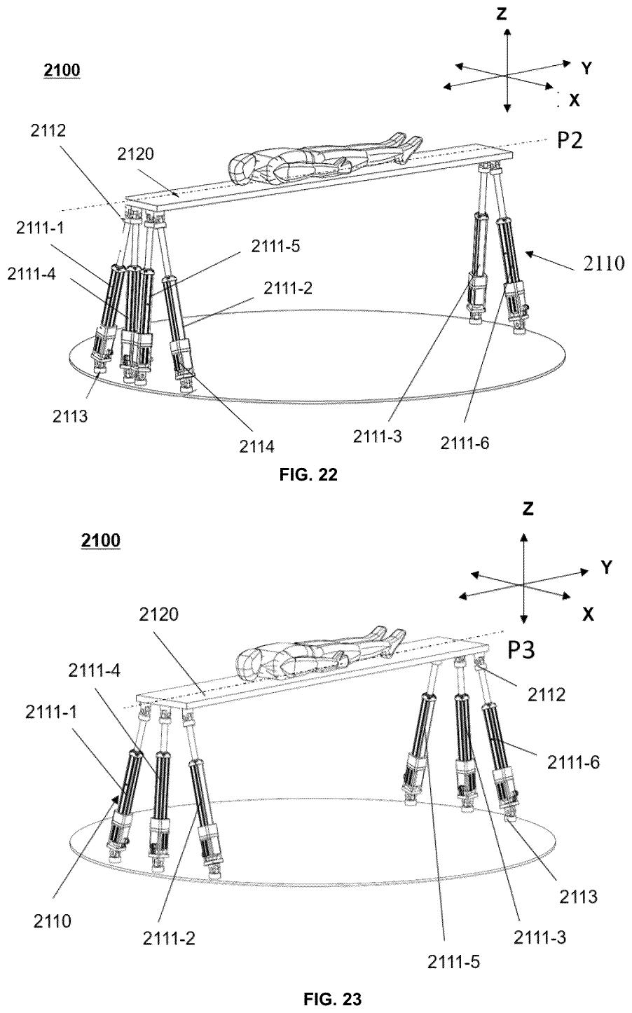

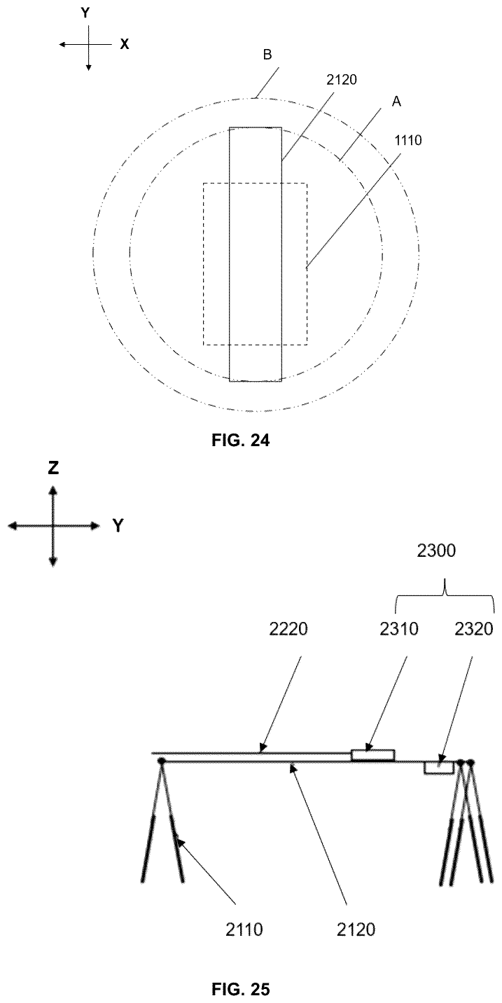

FIG. 22 is a schematic diagram illustrating a structure of the medical bed according to some embodiments of the present disclosure; FIG. 23 is a schematic diagram illustrating a structure of the medical bed according to some embodiments of the present disclosure; and FIG. 24 is a schematic diagram illustrating a structure of the medical bed according to some embodiments of the present disclosure.

In some embodiments, referring to FIGS. 22-24, the support device 2110 may include a plurality of telescopic rods 2111. One end of each of the plurality of telescopic rods 2111 may be connected to the medical bed board 2120 via a first hinge seat 2112. The other end of each of the plurality of telescopic rods 2111 may be connected to a static/fixed platform (e.g., the ground) via a second hinge seat 2113. In some embodiments, a portion of the plurality of telescopic rods 2111 may be connected to one end of the medical bed board 2120, and another portion of the plurality of telescopic rods 2111 may be connected to the other end of the medical bed board 2120. In some embodiments, a plurality of first hinge seats 2112 may be spaced apart along a first circumference, and a plurality of second hinge seats 2113 may be spaced apart along a second circumference.

In some embodiments, the support device 2110 may include at least two telescopic rods 2111. For example, two telescopic rods 2111 may be connected to the two ends of the medical bed board 2120 along the length direction of the medical bed board 2120, which solves the problem of sagging or bending of the cantilever bed plate 1212 in the cantilevered medical table 1210 due to the lack of support at one end, thus improving the safety during a medical process (e.g., radiographic imaging).

By hinging the plurality of telescopic rods 2111 to the medical bed board 2120 through the plurality of first hinge seats 2112 and to the static platform through the plurality of second hinge seats 2113, and by setting the count of the plurality of telescopic rods 2111 and the type and layout of the plurality of first hinge seats 2112 and the plurality of second hinge seats 2113, the medical bed 2100 can achieve multiple degrees of freedom in rotation and/or movement, meeting positioning requirements for subjects during medical operations.

In some embodiments, the support device 2110 may include at least three telescopic rods 2111. A portion of the at least three telescopic rods 2111 may be connected to one end of the medical bed 2120, and another portion of the at least three telescopic rods 2111 may be connected to the other end of the medical bed 2120.

In some embodiments of the present disclosure, the support device 2110 is provided with a plurality of telescopic rods 2111, each of which independently supports the medical bed board 2120, thereby preventing the accumulation of motion errors. In some embodiments of the present disclosure, the support device 2110 with the plurality of telescopic rods 2111 can also improve the stiffness of the medical bed 2100 and the precision of motion control.

In some embodiments, a count of the plurality of telescopic rods 2111 may be three, with one telescopic rod 2111 located at one end of the medical bed board 2120 and two telescopic rods 2111 located at the other end of the medical bed board 2120. Referring to FIG. 22, the at least three telescopic rods 2111 may include a first telescopic rod 2111-1, a second telescopic rod 2111-2, and a third telescopic rod 2111-3. A first end (an end near the medical bed board 2120) of each of the first telescopic rod 2111-1, the second telescopic rod 2111-2, and the third telescopic rod 2111-3 may be hinged to the medical bed board 2120 via a first hinge seat 2112. A second end (an end away from the medical bed board 2120) of each of the first telescopic rod 2111-1, the second telescopic rod 2111-2, and the third telescopic rod 2111-3 may be connected to a second hinge seat 2113. In some embodiments, the plurality of telescopic rods 2111 may be hinged to the static platform through a plurality of second hinge seats 2113. For example, the first telescopic rod 2111-1 and the second telescopic rod 2111-2 may be connected to one end of the medical bed board 2120 along a length direction of the medical bed board 2120 (i.e., a length direction of the medical bed 2100), and the third telescopic rod 2111-3 may be connected to the other end of the medical bed board 2120 along the length direction of the medical bed board 2120. FIG. 22 illustrates three orthogonal directions, wherein an X direction represents a width direction of the medical bed board 2120 (medical bed 2100), a Y direction represents a length direction of the medical bed board 2120 (medical bed 2100), and a Z direction represents a height direction of the medical bed board 2120.

By providing three telescopic rods 2111 on the medical bed 2100, with each telescopic rod 2111 hinged to the medical bed board 2120 through a first hinge seat 2112 and hinged to the static platform through a second hinge seat 2113, the medical bed 2100 may have six degrees of freedom. Through the telescopic motion of the three telescopic rods 2111, the medical bed board 2120 may translate along the X, Y, and Z directions, and/or rotate around the X, Y, and Z axes. It should be noted that the movements of the medical bed board 2120 in different degrees of freedom are achieved through the telescopic motion of the plurality of telescopic rods and the coordination between the plurality of first hinge seats 2112 and the plurality of second hinge seats 2113.

In some embodiments, the count of the plurality of telescopic rods 2111 may be six, and the medical bed 2100 may form a six-axis parallel platform. In some embodiments, the support device 2110 may include six telescopic rods 2111, where two telescopic rods 2111 are connected to one end of the medical bed board 2120, and four telescopic rods 2111 are connected to the other end of the medical bed board 2120; or, the supporting device 2110 includes six telescopic rods 2111, with three telescopic rods 2111 connected to one end of the medical bed board 2120, and three telescopic rods 2111 connected to the other end of the medical bed board 2120.

As shown in FIG. 23, six telescopic rods 2111 include: a first telescopic rod 2111-1, a second telescopic rod 2111-2, a third telescopic rod 2111-3, a fourth telescopic rod 2111-4, a fifth telescopic rod 2111-5, and a sixth telescopic rod 2111-6.

In some embodiments, referring to FIG. 23, two of the six telescopic rods 2111 (for example, the third telescopic rod 2111-3 and the sixth telescopic rod 2111-6) may be connected to one end of the medical bed board 2120 along the length direction of the medical bed board 2120, and the other four telescopic rods 2111 (for example, the first telescopic rod 2111-1, the second telescopic rod 2111-2, the fourth telescopic rod 2111-4, and the fifth telescopic rod 2111-5) may be connected to the other end of the medical bed board 2120 along the length direction of the medical bed board 2120. This arrangement provides more stable support for the medical bed board 2120 by the six telescopic rods 2111, enhancing the stability of the medical bed 2100. Additionally, the end of the medical bed 2100 with only two telescopic rods 2111 has more space due to the relatively fewer count of telescopic rods 2111, facilitating the positioning of the transfer bed 2200 (a bed used for transferring a subject) near the end with two telescopic rods 2111, thus facilitating the transfer of the subject between the transfer bed 2200 and the medical bed 2100.

In some embodiments, referring to FIG. 24, three of the six telescopic rods 2111 (for example, the first telescopic rod 2111-1, the second telescopic rod 2111-2, and the fourth telescopic rod 2111-4) may be connected to one end of the medical bed board 2120 along the length direction of the medical bed board 2120, and the other three telescopic rods 2111 (for example, the third telescopic rod 2111-3, the fifth telescopic rod 2111-5, and the sixth telescopic rod 2111-6) may be connected to the other end of the medical bed board 2120 along the length direction of the medical bed board 2120. This arrangement also enhances the stability of the medical bed 2100. It may be understood that when the support device 2110 includes six telescopic rods, the count of the plurality of first hinge seats 2112 and the count of the plurality of second hinge seats 2113 are also six, with the first end of each telescopic rod 2111 connected to a corresponding first hinge seat 2112 at the second end of each telescopic rod 2111 connected to a corresponding second hinge seat 2113.

By setting up six telescopic rods 2111 on the medical bed 2100, the medical bed 2100 may achieve six degrees of freedom. Through the telescopic motion of the three telescopic rods 2111, the medical bed board 2120 may translate along the X, Y, and Z directions, and rotate around the X, Y, and Z axes.

In some embodiments, the count of telescopic rods 2111 may be four, with one of the four telescopic rod 2111 connected to one end of the medical bed board 2120 along the length direction of the medical bed board 2120, and the other three of the four telescopic rods 2111 connected to the other end of the medical bed board 2120 along the length direction of the medical bed board 2120. Similar to the previous description, this configuration allows the medical bed 2100 to have six degrees of freedom. In some embodiments, the count of telescopic rods 2111 may be seven, with three of the seven telescopic rods 2111 connected to one end of the medical bed board 2120 along the length direction of the medical bed board 2120, and the other four of the seven telescopic rods 2111 connected to the other end of the medical bed board 2120 along the length direction of the medical bed board 2120. Similar to the previous description, this configuration allows the medical bed board 2100 to have six degrees of freedom. In some embodiments, the count of telescopic rods 2111 may be eight, with four of the eight telescopic rods 2111 connected to one end of the medical bed board 2120 along the length direction of the medical bed board 2120, and the other four of the eight telescopic rods 2111 connected to the other end of the medical bed board 2120 along the length direction of the medical bed board 2120. Similar to the previous description, this configuration allows the medical bed board 2100 to have six degrees of freedom.

In some embodiments, referring to FIGS. 22-24, at least two of the plurality of telescopic rods 2111 may be distributed on two sides of a central axis extending along the length direction of the medical bed board 2120. For example, as shown in FIG. 22, a first telescopic rod 2111-1 and a second telescopic rod 2111-2 may be distributed on the two sides of the central axis P1 extending along the length direction of the medical bed board 2120. A third telescopic rod 2111-3 may be precisely disposed on the central axis P1 or on either side of the central axis P1. As another example, as shown in FIG. 23, a third telescopic rod 2111-3 and a sixth telescopic rod 2111-6 may be distributed on the two sides of the central axis P2 extending along the length direction of the medical bed board 2120; a first telescopic rod 2111-1 and a second telescopic rod 2111-2 may be distributed on the sides of the central axis P2 extending along the length direction of the medical bed board 2120; a fourth telescopic rod 2111-4 and a fifth telescopic rod 2111-5 may be distributed on the two sides of the central axis P2 extending along the length direction of the medical bed board 2120. As yet another example, as shown in FIG. 24, a first telescopic rod 2111-1 and a second telescopic rod 2111-2 may be distributed on the two sides of the central axis P3 extending along the length direction of the medical bed board 2120; and a fifth telescopic rod 2111-5 and a sixth telescopic rod 2111-6 may be distributed on the two sides of the central axis P3 extending along the length direction of the medical bed board 2120. A third telescopic rod 2111-3 and a fourth telescopic rod 2111-4 may be precisely distributed on the central axis P3 or on either side of the central axis P3. In some embodiments, the two telescopic rods 2111 distributed on the two sides of the central axis extending along the length direction of the medical bed board 2120 may be symmetrically or asymmetrically distributed with respect to the central axis.

By arranging at least two telescopic rods on the two sides of the central axis extending along the length direction of the medical bed board 2120, the stability of the telescopic rods in supporting the medical bed board 2120 can be enhanced. In addition, the coordination between different telescopic rods enables the medical bed 2100 to move in multiple degrees of freedom, thus facilitating adjustment of a spatial position of the medical bed board 2120 and a position of the subject.

In some embodiments, each of the plurality of telescopic rods 2111 may be extended or retracted to adjust an overall length of the telescopic rod, and under the cooperation of the plurality of first hinge seats 2112 and the plurality of second hinge seats 2113, the medical bed 2100 may have multiple degrees of freedom (e.g., three degrees of freedom or six degrees of freedom), thereby facilitating the adjustment of the spatial position of the medical bed board 2120 and the position of the subject. Taking the third telescopic rod 2111-3 in FIG. 16 as an illustrative example, the third telescopic rod 2111-3 may include an inner rod and an outer sleeve, with the outer sleeve fitting over an outer side of the inner rod. One end of the inner rod may be hinged to the medical bed board 2120 through the first hinge seat 2112, and the other end of the inner rod may extend into the outer sleeve, with the outer sleeve connected to the second hinge seat 2113 away from the medical bed board 2120. The outer sleeve may be hinged to the static platform through the second hinge seat 2113. In some embodiments, the inner rod may be extended or retracted relative to the outer sleeve, and by controlling a length (also referred to as an extension length) of extension or retraction of the inner rod relative to the outer sleeve, the overall length of the third telescopic rod 2111-3 may be adjusted. In some embodiments, by adjusting the extension length of each of the plurality of telescopic rods separately to adjust the overall length of the telescopic rod and coordinating with the corresponding first hinge seat 2112 and the corresponding second hinge seat 2113, the medical bed 2100 may have different degrees of freedom, thus enabling the adjustment of the spatial position of the medical bed board 2120 and the position of the subject, thereby facilitating scanning and detection of the subject.

In some embodiments, each of the plurality of telescopic rods 2111 may be provided with a driving component (see FIG. 23) for driving the telescopic rod 2111 to extend and/or retract. In some embodiments, a count of the driving component 2114 may be one, and one driving component 2114 may be used for driving the telescopic rod 2111 to extend and retract. In some embodiments, the count of the driving component 2114 may be two, with one driving component 2114 configured to drive the telescopic rod 2111 to extend, and the other driving component 2114 configured to drive driving the telescopic rod 2111 to retract.

In some embodiments, the driving component 2114 may drive the inner rod of the corresponding telescopic rod 2111 to extend relative to the outer sleeve to increase the overall length of the telescopic rod 2111. As another example, the driving component 2114 may drive the inner rod of the corresponding telescopic rod 2111 to retract relative to the outer sleeve to decrease the overall length of the telescopic rod 2111. In some embodiments, the extension length of the inner rod relative to the outer sleeve of different telescopic rods 2111 may be reasonably set according to actual needs (e.g., a spatial position, a posture, etc., of the subject), and the extension or retraction of the plurality of telescopic rods 2111 driven by the corresponding driving component 2114 can be coordinated. In some embodiments, the driving component 2114 may include an electric cylinder, a pneumatic cylinder, a hydraulic cylinder, or the like. By providing the driving components 2114 to drive the plurality of telescopic rods 2111 to extend or retract respectively, an operation difficulty can be reduced for an operator, and the convenience and accuracy of operation can be improved.

FIG. 25 is a schematic diagram illustrating a structure of a first circumference and a second circumference according to some embodiments of the present disclosure. In some embodiments, referring to FIGS. 22-25, at least three first hinge seats 2112 may be spaced apart along a first circumference A, and at least three second hinge seats 2113 may be spaced apart along a second circumference B. In some embodiments, each hinge seat may include a hinge point. For example, referring to FIGS. 22 and 25, when the count of the plurality of telescopic rods is three, the three first hinge points at which the three first hinge seats 2112 are respectively located may form the first circumference A, and the three second hinge points at which the three second hinge seats 2113 are respectively located may form the second circumference B. In other words, the three first hinge seats 2112 are spaced apart along the first circumference A, and the three second hinge seats 2113 are spaced apart along the second circumference B. As another example, referring to FIG. 23 (or FIGS. 24) and 25, if the count of the plurality of telescopic rods is six, six first hinge seats 2112 are spaced apart along the first circumference A, and six second hinge seats 2113 are spaced apart along the second circumference B.

In some embodiments, the first end of each of the plurality of telescopic rods 2111 may be hinged to the medical bed board 2120 through the first hinge seat 2112 corresponding to the telescopic rod. When the telescopic rod extends or retracts under an action of a third driving member, the first hinge seat 2112 corresponding to the telescopic rod may move with the inner rod of the telescopic rod, causing a change in the position of the first hinge seat 2112, and thus a change in the hinge point corresponding to the first hinge seat 2112. Therefore, the first circumference A may also be referred to as a dynamic circumference. The second end of each of the plurality of telescopic rods may be hinged to the static platform through the corresponding second hinge seat 2113, and a position of the second hinge seat 2113 is fixed. Therefore, the second circumference B may also be referred to as a static circumference.

By arranging a positional distribution of the plurality of first hinge seats 2112 on the first circumference A and a positional distribution of the plurality of second hinge seats 2113 on the second circumference B, a movement of the medical bed board 2120 of the medical bed 2100 in multiple degrees of freedom within an aperture 105 of a scanning device (e.g., a movement along the length direction of the medical bed board 2120 or in a direction perpendicular to the length direction of the medical bed board 2120) can be controlled.

It should be noted that the illustration in FIG. 25, where a diameter of the first circumference A is smaller than a diameter of the second circumference B, is merely exemplary. In actual situations, the diameter of the first circumference A may also be equal to or greater than the diameter of the second circumference B. The present disclosure does not specifically limit a size relationship between the diameter of the first circumference A and the diameter of the second circumference B.

In some embodiments, the first hinge seat 2112 may include a Hooke joint or a ball joint, and the second hinge seat 2113 may include a Hooke joint. In some embodiments, a type of the first hinge seat 2112 and a type of the second hinge seat 2113 may be the same. For example, both the first hinge seat 2112 and the second hinge seat 2113 may be Hooke joints. In some embodiments, the type of the first hinge seat 2112 and the second hinge seat 2113 may be different. For example, the first hinge seat 2112 may be a ball joint, and the second hinge seat 2113 may be a Hooke joint. By setting the first hinge seat 2112 as a Hooke joint or a ball joint and the second hinge seat 2113 as a Hooke joint, the medical bed board 2120 can move in multiple degrees of freedom, and the stability of the medical bed 2100 can be maintained even after the medical bed board 2120 moves in different degrees of freedom (i.e., a stable state of the medical bed 2100 remains unchanged after movement of the medical bed board 2120).

In some embodiments, one end of each of the plurality of telescopic rods 2111 may be hinged to the medical bed board 2120 through a Hooke joint or a ball joint, and the other end of each of the plurality of telescopic rods 2111 may be hinged to the static platform through a Hooke joint. By adjusting the count of the plurality of telescopic rods, the medical bed 2100 may have different degrees of freedom. For example, if the count of the plurality of telescopic rods is six, the medical bed 2100 may have six degrees of freedom. As another example, if the count of the plurality of telescopic rods is three, the medical bed 2100 may have three degrees of freedom. Furthermore, compared to a serial structure multi-degree-of-freedom platform, the parallel structure multi-degree-of-freedom medical bed 2100 provided in the embodiments of the present disclosure has greater stiffness, load-bearing capacity, and higher motion control accuracy by adjusting the count of the plurality of telescopic rods, the types of the plurality of first hinge seat 2112 and the plurality of second hinge seat 2113, and the positional distributions of the plurality of first hinge seats 2112 and the plurality of second hinge seats 2113. Additionally, if the serial structure multi-degree-of-freedom platform has more degrees of freedom, a weight of the serial structure is increased, resulting in greater inertia of the platform. In contrast, the parallel structure multi-degree-of-freedom medical bed 2100 has the advantage of achieving smaller inertia.

In some embodiments, the medical bed 2100 may further include a plurality of telescopic guardrails, which may be located around a perimeter of the medical bed board 2120.

Telescopic guardrails refer to guardrails with telescopic functionality. In some embodiments, the plurality of telescopic guardrails may extend along a direction perpendicular to an upper surface of the medical bed board 2120 (e.g., the Z-direction in FIG. 2). In some embodiments, when the movable bed board 2220 moves onto the medical bed board 2120, the plurality of telescopic guardrails may extend upward along a direction perpendicular to the upper surface of the medical bed board 2120 (e.g., the Z-direction in FIG. 2) until they surpass the upper surface of the movable bed board 2220. By providing the plurality of telescopic guardrails, they may be extended when needed to protect a subject on the movable bed board 2220, preventing the subject from falling off.

In some embodiments, a flexible component may be provided on a side of the plurality of telescopic guardrails close to the subject.

The flexible component refers to a structure made of a flexible material, for example, rubber, cotton, sponge, air cushions, etc. By providing the flexible component on the side of the plurality of telescopic guardrails close to the subject, the flexible component may act as a buffer when the subject collides with the plurality of telescopic guardrails, thereby improving subject comfort.

In some embodiments, the plurality of telescopic guardrails may be configured to: in response to receiving a guardrail activation instruction, activate the corresponding telescopic guardrail. In some embodiments, the plurality of telescopic guardrails may be connected to a control device of the medical bed apparatus 2000 via a signal. The control device of the medical bed apparatus 2000 may generate the guardrail activation instruction and send the guardrail activation instruction to the plurality of telescopic guardrails, causing at least one of the plurality of telescopic guardrails to execute at least one action of extending upward, retracting downward, etc.

In some embodiments, the guardrail activation instruction may be determined based on subject characteristics, an enabled degree of freedom, and a medical aperture.

The subject characteristics refer to characteristics related to the subject. In some embodiments, the subject characteristics may include at least one of a height, a weight, an age, etc. of the subject. The subject characteristics may be manually obtained and input into the control device of the medical bed apparatus 2000.

The enabled degree of freedom refers to a direction in which the medical bed board 2120 needs to move and/or rotate. For example, the medical bed board 2120 may move along a length direction (e.g., the Y-direction in FIG. 2). As another example, the medical bed board 2120 may rotate around a vertical axis (e.g., the Z-direction in FIG. 2). The control device of the medical bed apparatus 2000 may determine the enabled degree of freedom by monitoring the movement of the medical bed board 2120 through a displacement sensor, etc.

The medical aperture refers to an aperture of the medical space 1110. In some embodiments, the control device of the medical bed apparatus 2000 may obtain the medical aperture in various ways. For example, the control device may obtain the medical aperture based on at least one of a manufacturer, historical data, manual input, etc.

The guardrail activation instruction may include the telescopic guardrail(s) to be activated, a height of extension or retraction, and a speed of extension or retraction. In some embodiments, the control device of the medical bed apparatus 2000 may determine the telescopic guardrail(s) to be activated based on the enabled degree of freedom and determine the height of extension or retraction of the telescopic guardrail(s) based on the subject characteristic, and the speed of extension or retraction may be preset.

For example, if the enabled degree of freedom is rotating to one side around the Y-axis (as flipping outwards in FIG. 16), then the telescopic guardrail to be activated may be the telescopic guardrail on a side of a rotation direction (e.g., closer to the outer side of the paper in FIG. 16).

In some embodiments, a correspondence table between the subject characteristics and the height of extension or retraction may be constructed. For example, by statistically analyzing average dimensional information of subjects in subject characteristics (e.g., an average body dimension of 12-year-olds weighing 30 kg and 140 cm tall), the required height of extension or retraction may be manually determined and recorded in a table to form the correspondence table. It may be understandable that since the aperture of the medical space 1110 is fixed, the height of extension or retraction may be limited by the aperture of the medical space 1110, i.e., the height of extension or retraction of the telescopic guardrails may not exceed a set height threshold. If the height of extension or retraction is less than the aperture of the medical space 1110, then the height of extension or retraction may be determined as valid. If the height of extension or retraction exceeds the aperture of the medical space 1110, then the height of extension or retraction may be determined as invalid, and in this case, the actual the height of extension or retraction of the telescopic guardrails may be the height threshold.

In some embodiments, by providing the plurality of telescopic guardrails, automated control of the plurality of telescopic guardrails can be achieved when the medical bed board moves and/or rotates, thus protecting the subject and preventing the subject from falling off the movable bed board.

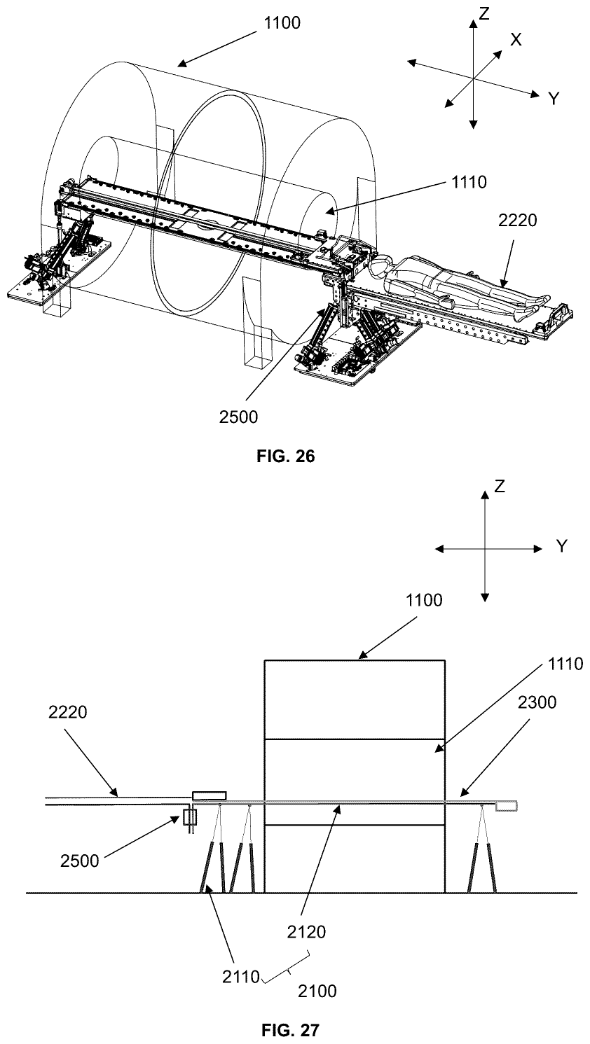

FIG. 26 is a schematic diagram illustrating a structure of the conveying device according to some embodiments of the present disclosure. In some embodiments, as shown in FIG. 26, the conveying device 2300 includes the transfer member 2310 and a drive device 2320. The drive device 2320 may be configured to drive the transfer member 2310 to move along a length direction of the medical bed board 2120. The transfer member 2310 may be configured to drag the movable bed board 2220, directly pulling the movable bed board 2220 and a subject on the movable bed board 2220 onto the medical bed board 2120. For example, the transfer member 2310 may be a trailer. In some embodiments, the movable bed board 2220 may be part of a transfer bed (e.g., the transfer bed 2200), and the movable bed board 2220 may be detachably provided on a movable bed body (e.g., the movable bed body 2210). The movable bed body may drive the movable bed board 2220 and the subject on the movable bed board 2220 to a position around the medical bed 2100 (e.g., to an end of two telescopic rods of a transfer bed with six telescopic rods). Then the movable bed body may dock with the transfer member 2310 and detach from the movable bed body under the drag of the transfer member 2310 (e.g., the trailer). The transfer member 2310 may drive the movable bed board 2220 to move onto the medical bed board 2120. In some embodiments, the movable bed board 2220 may be carried by medical staff to a position around the medical bed 2100 and docked with the transfer member 2310 (e.g., the trailer). The arrangement of the transfer member 2310 and the drive device 2320 can transport the subject over a longer distance.

In some embodiments, the transfer member 2310 and the drive device 2320 may be connected in a transmission way through a transmission assembly. In some embodiments, the transmission assembly may include a belt drive mechanism 2331 or a chain drive mechanism 2332 (see FIG. 13). In some embodiments, the transmission assembly may include a gear rack transmission assembly or a worm gear transmission assembly.

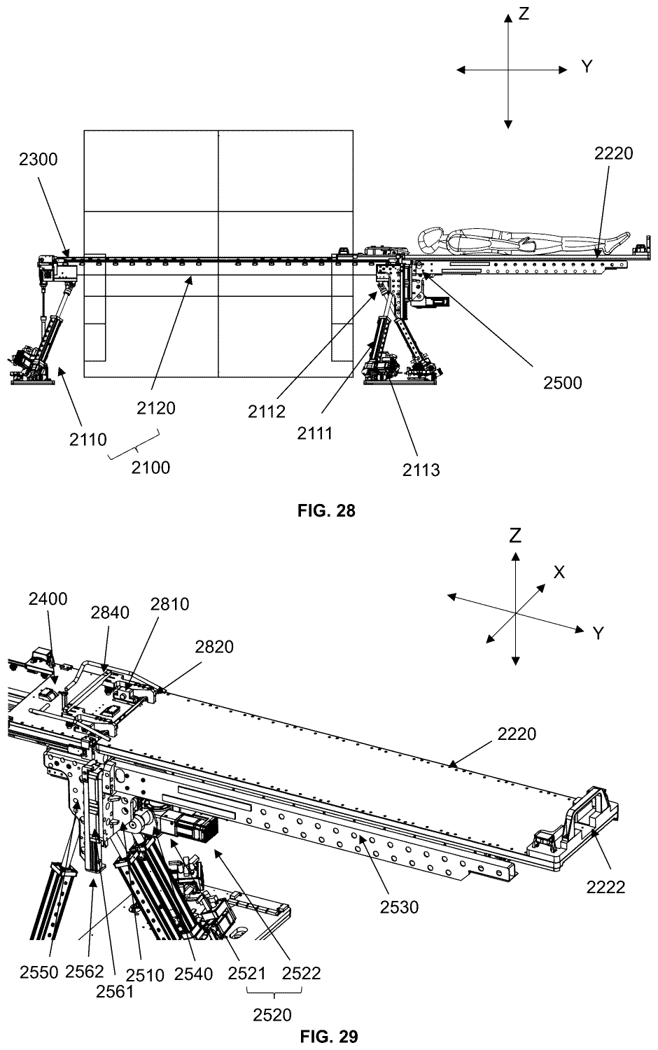

FIG. 27 is a schematic diagram illustrating a structure of a medical system according to some embodiments of the present disclosure; FIG. 28 is a schematic diagram illustrating a structure of the medical bed apparatus according to some embodiments of the present disclosure; and FIG. 29 is a schematic diagram illustrating a structure of a medical bed apparatus according to some embodiments of the present disclosure.

In some embodiments, as shown in FIG. 27, the medical bed apparatus 2000 further includes a lifting platform 2500, and the lifting platform 2500 is disposed on the medical bed 2100 and can be raised and lowered.

In some embodiments, a movable bed board 2220 is detachably disposed on the lifting platform 2500; and a conveying device 2300 transfers, on the medical bed 2100, the movable bed board 2220 detached from the lifting platform 2500.

The lifting platform 2500 refers to a platform capable of being raised or lowered relative to the medical bed 2100. The lifting platform 2500 is connected to the medical bed 2100 and can be raised and lowered. For example, the lifting platform 2500 is attached to one end of the medical bed 2100 along its length direction. The elevation of the lifting platform 2500 can be understood as a change in its height relative to the ground. For example, the lifting platform 2500 may be raised and lowered along the Z-axis of FIGS. 27-29. The lifting platform 2500 may be located outside of the medical space 1110, at which point the lifting of the lifting platform 2500 may be unrestricted by the dimensions of the medical space 1110.

In some embodiments, prior to the start of an operation, the lifting platform 2500 is lowered as shown in FIG. 27, facilitating the subject to get onto the lifting platform 2500, and the lower height facilitates the doctor's associated operation on the subject. The lifting platform 2500 then rises, until it is substantially the same height as the medical bed 2100 (as shown in FIG. 28), and the subject is transferred inside the medical space 1110 (e.g., via the conveying device), as shown in FIG. 35. After the operation is completed, the subject is transferred back to the lifting platform 2500 from the medical bed 2100 until it is moved out of the medical space 1110, and the lifting platform 2500 is lowered to facilitate dismounting of the subject.

In some embodiments, the movable bed board 2220 may transfer the subject between the lifting platform 2500 and the medical bed 2100. The fact that the movable bed board 2220 is detachably disposed on the lifting platform 2500 may be understood to mean that the movable bed board 2220 is disposed on the lifting platform 2500, but that the movable bed board 2220 may be removed from the lifting platform 2500. For example, the movable bed board 2220 may be connected to the lifting platform 2500 in a detachable connection. As another example, the movable bed board 2220 may be placed directly on the lifting platform 2500 (the movable bed board 2220 and the lifting platform 2500 may not be connected). When the movable bed board 2220 is placed on the lifting platform 2500, the movable bed board 2220 may be removed from the lifting platform 2500 in one or more directions (e.g., lifting the movable bed board 2220 upward may remove it from the lifting platform 2500).

In some embodiments, the conveying device 2300 transfers, on the medical bed 2100, the movable bed board 2200 disconnected from the lifting platform 2400. For example, the conveying device 2300 may transfer, to the medical bed 2100, the movable bed board 2200 that is disconnected from the lifting platform 2400.

In some embodiments, the lifting platform 2400 is connected to the medical bed board 2110 and can be raised and lowered.

In some embodiments of the present disclosure, by setting up the medical bed apparatus including the medical bed and the lifting platform, the integration of the device is improved, and at the same time, the lifting and lowering can be carried out on a wider range as compared to the setup of lifting and lowering the medical bed directly, which makes it easier for the subject to get on and off the bed and for the physician maneuvering.

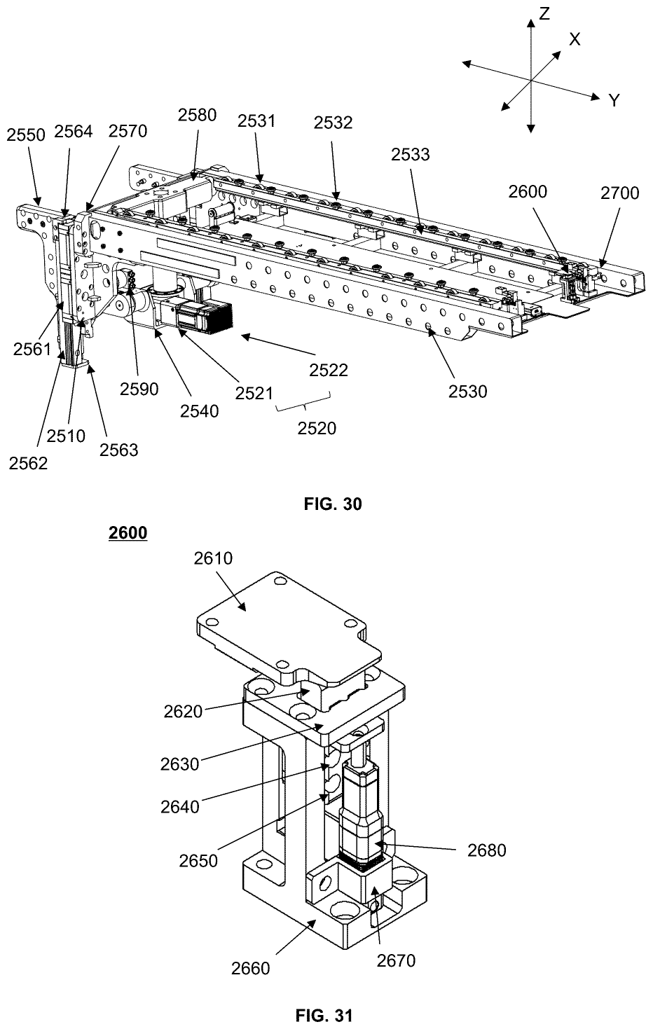

FIG. 30 is a schematic diagram illustrating a structure of a lifting platform with a docking assembly according to some embodiments of the present disclosure; and FIG. 31 is a schematic diagram illustrating a structure of a lifting platform according to some embodiments of the present disclosure.

In some embodiments, the lifting platform 2500 includes a lifting seat 2510, a lifting drive mechanism 2520, and a bed frame 2530 as shown in FIGS. 30-31. The bed frame 2530 is fixed to the lifting seat 2510, and the movable bed board 2220 is detachably provided on the bed frame 2530. The lifting seat 2510 is connected to the medical bed 2100. The lifting drive mechanism 2520 is drive connected to the lifting seat 2510.

The lifting seat 2510 refers to a structure for supporting the bed frame 2530. In some embodiments, the lifting seat 2510 may be connected to the medical bed 2100 via a connection base 2550. The connection base 2550 is configured to connect the lifting platform 2500 to the medical bed 2100. In some embodiments, the connection base 2550 may include a first connecting portion and a second connecting portion connected to each other, where the first connecting portion may be connected to the medical bed board 2120, and the second connecting portion may be connected to the lifting seat 2510.

The lifting drive mechanism 2520 refers to a mechanism for driving the lifting seat 2510 for raising and lowering. In some embodiments, the lifting drive mechanism 2520 may be mounted to a drive base 2540.

In some embodiments, the lifting drive mechanism 2520 may include a lifting transmission mechanism 2521 and a drive source 2522, as shown in FIGS. 30-31. In some embodiments, the drive source 2522 may be a motor, a hydraulic cylinder, a pneumatic cylinder, or the like. The lifting drive mechanism 2520 is connected to and configured to drive the lifting seat 2510. The lifting transmission mechanism 2521 may transfer power from the drive source 2522 to the lifting seat 2510.

The bed frame 2530 refers to a structure for supporting the movable bed board 2220. In some embodiments, the movable bed board 2220 may be disengaged from the bed frame 2530 to enable the bed frame 2530 to be transferred by the conveying device 2300.

In some embodiments, the lifting platform 2500 may further include a bed frame fixing base 2570 and a drive lifting seat 2580. The bed frame fixing base 2570 and the drive lifting seat 2580 are configured to realize the fixing of the bed frame 2530 to the lifting seat 2510. In some embodiments, the bed frame fixing base 2570 is fixedly connected to the bed frame 2530, and both the bed frame fixing base 2570 and the drive lifting seat 2580 are fixedly connected to the lifting seat, to realize that the lifting process of the bed frame 2530 is fixed relative to the lifting seat 2510.

In some embodiments, the lifting platform 2500 further includes an encoder 2590 for feedback of a position of the lifting platform 2500 during lifting, as shown in FIG. 31.

In some embodiments of the present disclosure, the lifting and lowering process can be made more flexible by providing the lifting platform including the lifting seat, the lifting drive mechanism, and the bed frame.

In some embodiments, the lifting drive mechanism 2520 includes a drive motor, a screw, and a nut. The drive motor rotates with the drive screw. The nut is socketed on the screw. The nut is rotatably disposed on the lifting seat 2510.

The drive motor is configured to power the lifting process. Specifically, the drive motor may drive the screw to rotate.

The screw and the nut are configured to convert the power supplied by the drive motor into motion in the vertical direction (e.g., the Z-axis direction). In some embodiments, the nut is socketed on the screw, and the nut is rotatably disposed on the lifting seat 2510.

In some embodiments, the drive motor may drive the screw to rotate, causing the nut to move up and down along the screw, which in turn drives the lifting seat 2510 up and down.

In some other embodiments, the lifting drive mechanism 2520 may further include the drive motor, a rack, and a gear; the drive motor drives the gear to rotate; the gear engages the rack; and the gear is rotatably disposed on the lifting seat 2510. In some embodiments, the drive motor realizes the lifting and lowering of the lifting seat 2510 by driving the gear to reciprocate on the rack. In some embodiments, the lifting drive mechanism 2520 further includes a speed reducer and a synchronous belt drive mechanism, where the drive motor and the gears may be connected via the speed reducer and the synchronous belt drive mechanism. The gear reducer is configured to match the rotational speed of the drive motor and the synchronous belt drive mechanism. The synchronous belt drive mechanism is configured to transmit torque to the gears. In some embodiments, the drive motor can output torque and rotation speed, which is transmitted by the synchronous belt drive mechanism to drive the gears on the rack-and-pinion mechanism to reciprocate on the racks, and drive the lifting platform 2500 to lift.

In some embodiments of the present disclosure, by setting the lifting drive mechanism to include a drive motor, a screw, and a nut, or by setting the lifting drive mechanism to include a drive motor, a rack, and a gear, it can be ensured that the lifting platform can be elevated and lowered stably.

In some embodiments, as shown in FIGS. 30-31, the medical bed apparatus 2000 further includes a slider 2561 fixed to the lifting seat 2510 and a guide rail 2562 fixed to the medical bed 2100.

The slider 2561 and the guide rail 2562 are configured to guide the lifting and lowering of the lifting seat 2510. In some embodiments, the slider 2561 moves along the guide rail 2562 during the lifting and lowering of the lifting seat 2510. In some embodiments, the guide rail 2562 is provided with a slider lower limit 2563 at a lower end and a slider upper limit 2564 at an upper end, as shown in FIG. 30. The slider lower limit 2563 and the slider upper limit 2564 are configured to limit the height of lifting and to prevent the slider 2561 from detaching from the guide rail 2562.

In some embodiments of the present disclosure, by setting the slider and the guide rail, the lifting platform can be guided so that the lifting and lowering process is smoother and more solid.

In some embodiments, the bed frame 2530 is provided with a first roller set, as shown in FIG. 31, and the first roller set is configured to reduce the friction between the movable bed board 2220 and the bed frame 2530. The first roller set includes a plurality of first rollers 2531 spaced apart along the conveying direction of the conveying device 2300, and an outer peripheral surface of the first roller 2531 is able to contact a bottom of the movable bed board 2220. The conveying direction of the conveying device 2300 is the direction in which the conveying device 2300 moves on the medical bed 2100 (e.g., the Y-axis direction in FIGS. 27-31).

In some embodiments, the bed frame 2530 is provided with a second roller set, as shown in FIG. 31, and the second roller set is configured to guide the movable bed board 2220. The second roller set includes a plurality of second rollers 2532 spaced apart along the conveying direction of the conveying device 2300. In some embodiments, the bottom of the movable bed board 2220 is provided with a guide groove (not shown in the drawings) extending in the length direction, and an outer peripheral surface of the second roller 2532 is capable of contacting a sidewall of the guide groove.

In some embodiments, the second rollers 2532 are disposed on the limiting rail 2533 that extends along the length direction of the bed frame 2530, and the limiting rail 2533 may cooperate with a guide groove of the movable bed board 2220. The length direction of the bed frame 2530 may also be equivalent to the conveying direction of the conveying device 2300.

In some embodiments of the present disclosure, smooth sliding of the movable bed board on the bed frame can be ensured by providing the first roller set on the bed frame. By providing a second roller set on the bed frame, the smooth sliding of the movable bed board on the bed frame can be ensured and guided by cooperating with the guiding groove to realize guiding to avoid the deviation of the movable bed board.

When the bed frame 2530 is connected to the lifting seat 2510 at only one end, when the subject gets on and off the bed, if the center of gravity of the subject is at the end of the bed frame 2530 that is far away from the lifting seat 2510, it may lead to an uneven force on the bed frame 2530. In some embodiments, the end of the bed frame 2530 away from the lifting seat 2510 may be provided with a support bar that is folded with respect to the bed frame 2530, so that the support bar may be folded and stowed away during lifting, and can be unfolded when the subject gets in and out of the bed to provide more stable support for the bed frame 2530. The support bar is capable of being driven by a drive device to telescope to adjust the length to accommodate different heights.

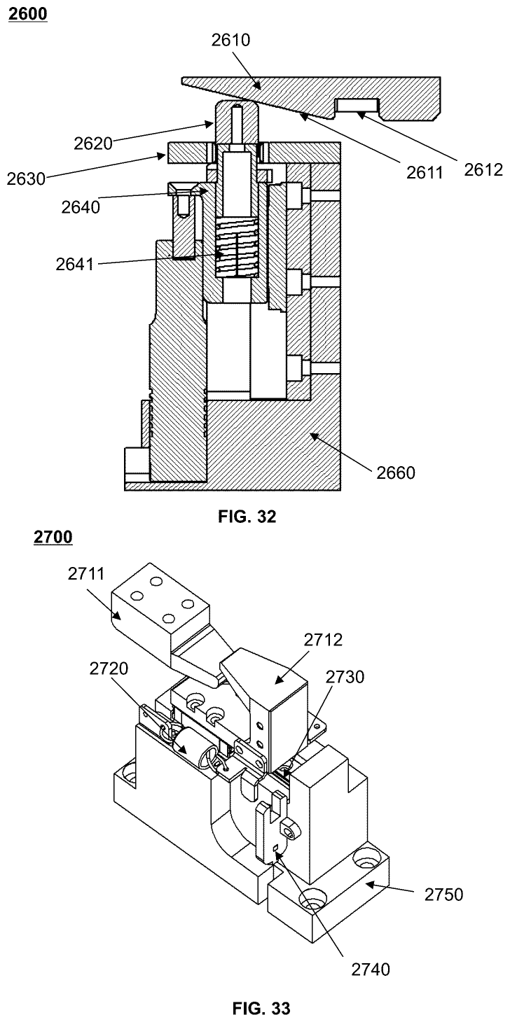

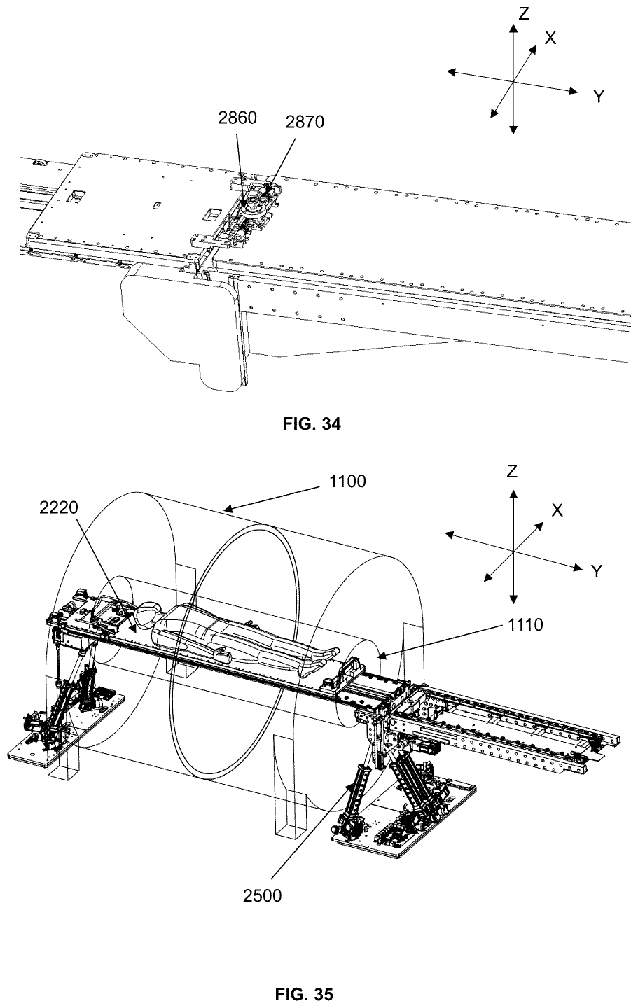

FIG. 32 is a schematic diagram illustrating a structure of a bed plate locking mechanism and a bed plate limiting mechanism according to some embodiments of the present disclosure; FIG. 33 is a schematic diagram illustrating an internal structure of the bed plate locking mechanism according to some embodiments of the present disclosure; and FIG. 34 is a schematic diagram illustrating a structure of a bed plate limiting mechanism according to some embodiments of the present disclosure.

In some embodiments, as shown in FIGS. 31-32, the medical bed apparatus 2000 further includes a bed plate locking mechanism 2600, and the bed plate locking mechanism 2600 is configured to lock the movable bed board 2220 to the bed frame 2530.

In some embodiments, as shown in FIGS. 31-33, the medical bed apparatus 2000 further includes a bed plate limiting mechanism 2700, and the bed plate limiting mechanism 2700 is configured to limit the distance of movement of the movable bed board 2220 in a direction away from the medical bed 2100. In some embodiments, the bed plate limiting mechanism 2700 may be provided with two, and the two bed plate limiting mechanisms 2700 may be symmetrically disposed along the X-axis direction based on a central axis of the bed frame 2530.

In some embodiments, as shown in FIG. 30, a bed plate handle 2222 is provided at one end of the movable bed board 2220 to facilitate an operator (e.g., a physician) to adjust the position of the movable bed board 2220. In some embodiments, the bed plate limiting mechanism 2700 may cooperate with the bed plate handle 2222 to limit the movable bed board 2220.

When the movable bed board 2220 is transferred to the lifting platform 2500, the two bed plate limiting mechanisms 2700 may limit the displacement of the bed plate in the lateral (Y-axis) and vertical (Z-axis) directions, and the bed plate locking mechanism 2600 may lock the lateral (Y-axis) position of the movable bed board 2220 to keep the initial position of the movable bed board 2220 stable.

In some embodiments of the present disclosure, by providing a bed plate locking mechanism and/or a bed plate limiting mechanism, it is possible to ensure that the movable bed board is stably connected to the bed frame when it is not disengaged.

In some embodiments, as shown in FIGS. 32-33, the bed plate locking mechanism 2600 includes a bed plate locking base 2610, a bed plate locking block 2620, and a locking drive mechanism 2680; the bed plate locking base 2610 is fixedly disposed on the movable bed board 2220; the bed plate locking base 2610 is provided with a locking hole 2612; the locking drive mechanism 2680 is disposed on the bed frame 2530, and the locking drive mechanism 2680 is connected to and configured to drive the bed plate locking block 2620. In some embodiments, the locking drive mechanism 2680 actuates the bed plate locking block 2620 in a direction that extends into or out of the locking hole 2612. The locking hole 2612 may be a groove in the bottom of bed plate locking base 2610 that is shaped to cooperate with at least a portion of bed plate locking block 2620. The bed plate locking block 2620 can lock the movable bed board 2220 in conjunction with the locking hole 2612.

In some embodiments, the bed plate locking block 2620 may be in a state capable of cooperating with the locking hole 2612 when the locking drive mechanism 2680 drives the bed plate locking block 2620 in a direction that extends into the locking hole 2612. For example, when the bed plate locking base 2610 drives the locking hole 2612 to a position corresponding to the bed plate locking block 2620, the bed plate locking block 2620 may be further driven by the locking drive mechanism 2680 to into the locking hole 2612. As another example, when the bed plate locking base 2610 moves the locking hole 2612 to a position corresponding to the bed plate locking block 2620, the bed plate locking block 2620 may be extended into the locking hole 2612 under the action of some other mechanism, such as the first elastic component 2641 as described below. When it is necessary to disengage the locking, the locking drive mechanism 2680 may actuate the bed plate locking block 2620 in a direction away from the locking hole 2612 to cause the bed plate locking block 2620 to disengage from the locking hole 2612.

In some embodiments, the locking drive mechanism 2680 includes a locking motor. In some other embodiments, the locking drive mechanism 2680 may also include a hydraulic cylinder, a pneumatic cylinder, or the like.

In some embodiments, the bed plate locking mechanism 2600 further includes a locking limit block 2630, a locking slider guide 2650, and a locking assembly base 2660.

The locking limit block 2630 refers to a structure for limiting the bed plate locking block 2620. In some embodiments, the locking limit block 2630 is provided with a hole through which the bed plate locking block 2620 may extend or retract. The locking slider guide 2650 is configured to guide the lifting and lowering of a locking lift block 2640. In some embodiments, the locking lift block 2640 may slide up and down along the locking slider guide 2650 to drive the bed plate locking block 2620 for locking and unlocking the bed plate locking mechanism 2600. The locking assembly base 2660 is configured to secure or carry the various structures in the bed plate locking mechanism 2600 other than the bed plate locking base 2610.

In some embodiments, the locking assembly base 2660 is provided with a locking motor base 2670, and in some embodiments, the locking motor is fixedly disposed on the locking motor base 2670.

In some embodiments, as shown in FIG. 33, the bed plate locking mechanism 2600 further includes a locking ramp 2611, the locking lifting block 2640, and the first elastic component 2641; the locking ramp 2611 is disposed on the bed plate locking base 2610, and the locking hole 2612 is disposed at an end of the locking ramp 2611 near the medical bed 2100; the locking lifting block 2640 is connected to the locking drive mechanism 2680; the bed plate locking block 2620 is slidably disposed on the locking lifting block 2640 along the direction of extending into or out of the locking hole 2612; and the first elastic component 2641 is disposed between the bed plate locking block 2620 and the locking lifting block 2640. In some embodiments, the first elastic component 2641 is a spring, rubber, or the like that resists the bed plate locking block 2620 at the top end and resists the locking lift block 2640 at the bottom end.

In some embodiments, the locking ramp 2611 refers to a beveled surface on the underside of the bed plate locking base 2610 that is sloped downwardly from the end facing toward the medical bed 2100 toward the end facing away from the medical bed 2100, as illustrated in FIG. 33.

In some embodiments, the locking lift block 2640 is configured to transmit a driving force of the locking drive mechanism 2680. For example, the locking drive mechanism 2680 may drive the locking lift block 2640 in a direction that extends into or out of the locking hole 2612 to drive the bed plate locking block 2620 in a direction that extends into or out of the locking hole 2612.

In some embodiments, when locking is required, the locking drive mechanism 2680 actuates the locking lift block 2640 in the direction of extending into the locking hole 2612, and the bed plate locking block 2620 may be in a state capable of mating with the locking aperture 2612. The movable bed board 2220 drives the bed plate locking base 2610 to trigger the bed plate locking block 2620 to slide downward along the locking ramp 2611, and with the lowering of the locking ramp 2611 causing the first elastic component 2641 to undergo an elastic deformation, and the first elastic component 2641 springs back after the bed plate locking base 2610 moves to a position corresponding to the bed plate locking block 2620, and under the elastic return force of the first elastic component 2641, the bed plate locking block 2620 passes through the hole in the locking limit block 2630 and extends into the locking hole 2612 to perform locking. When it is necessary to unlock, the locking drive mechanism 2680 drives the bed plate locking block 2620 in the direction away from the locking hole 2612 by driving the locking lifting block 2640 down until it is moved out of the locking hole 2612, thereby unlocking the bed plate locking mechanism 2600, and releasing the movable bed board 2220. When it is necessary to lock again, the locking drive mechanism 2680 may drive the bed plate locking block 2620 to move in the direction of extending into the bed plate locking block 2620 until the bed plate locking block 2620 extends into the locking hole 2612.

In some embodiments, as shown in FIG. 34, the bed plate limiting mechanism 2700 includes a first limiting block 2711, a second limiting block 2712, a limiting block mechanism, a limiting rail 2730, and a limiting base 2750; the first limiting block 2711 is fixedly disposed on the movable bed board 2220; the second limiting block 2712 is capable of abutting against the first limiting block 2711 during the movement of the movable bed board 2220 away from the medical bed 2100, and the second limiting block 2712 is slidably disposed on the limiting rail 2730; and the limiting rail 2730 is disposed on the limiting base 2750, and the limiting block mechanism restricts the second limiting block 2712 to move on the limiting rail 2730 in a direction away from the medical bed 2100.

The first limiting block 2711 is configured to cooperate with the second limiting block 2712 in limiting displacement of the movable bed board 2220. In some embodiments, the first limiting block 2711 is provided at an end of the movable bed board 2220 away from the medical bed 2100, and the second limiting block 2712 is able to press against the movable bed board 2220 during movement of the first limiting block 2712 to realize the limitation of the movable bed board 2220. The limiting block mechanism is configured to limit the movement distance of the second limiting block 2712. In some embodiments, the limiting block mechanism may be a blocking block provided at the end of the limiting rail 2730 toward the end of the direction away from the medical bed 2100. In some embodiments, the limiting block mechanism may also be a pull cord provided on the second limiting block 2712, and when the second limiting block 2712 is moved a certain distance toward away from the medical bed 2100, the pull cord is tautened to restrict further movement of the second limiting block 2712. The limiting rail 2730 is configured to guide the displacement of the second limiting block 2712. In some embodiments, the limiting rail 2730 extends in a direction consistent with the conveyance direction of the movable bed board 2220. In some embodiments, when the first limiting block 2711 rests against the second limiting block 2712 and slides along the limiting rail 2730 to a limit position, the limiting of the movable bed board 2220 is completed. The limit position refers to a position where the limiting block mechanism restricts the second limiting block 2712 from continuing to move. The limiting base 2750 is configured to secure or carry a plurality of structures in the bed plate limiting mechanism 2700.

In some embodiments, as shown in FIG. 34, the bed plate limiting mechanism 2700 is further provided with a second elastic component 2720, the second elastic component 2720 is disposed between the second limiting block 2712 and the limiting base 2750, and when the second limiting block 2712 moves in a direction away from the medical bed 2100 on the limiting rail 2730, the second elastic component 2720 undergoes elastic deformation. In some embodiments, one end of the second elastic component 2720 proximate the medical bed 2100 is coupled to the limiting base 2750, and one end of the second elastic component 2720 proximate the medical bed 2100 is coupled to the second limiting block 2712. The second elastic component 2720 may be a spring, rubber, etc. The second elastic component 2720 may play a limiting role and a cushioning role for the second limiting block 2712 when the second limiting block 2712 moves on the limiting rail 2730 in a direction away from the medical bed 2100.

In some embodiments, the movable bed board 2220 drives the first limiting block 2711 against the second limiting block 2712 when moving in a direction away from the medical bed 2100, and moves along the limiting rail 2730 to a limiting position, the second elastic component 2720 is stretched to produce an elastic deformation, and the displacement of the movable bed board 2220 in the direction away from the medical bed 2100 is limited. The movement of the movable bed board 2220 in the direction of the medical bed 2100 leads to the separation of the first limiting block 2711 from the second limiting block 2712, under the action of the second elastic component 2720, the second limiting block 2712 is reset along the limiting rail 2730.

In some embodiments, the bed plate limiting mechanism 2700 is further provided with an in-place sensor 2740 for detecting whether the first limiting block 2711 and the second limiting block 2712 are pressed against each other and moved to the limiting position. In some embodiments, the in-place sensor 2740 is signaling connected to the lifting drive mechanism 2520, and after the in-place sensor 2740 detects that the first limiting block 2711 is pressed against and moves to the second limiting block 2712 to the limit position, then the lifting and lowering operation is implemented, thereby ensuring safety.

In some embodiments of the present disclosure, by setting up the bed plate locking mechanism and the bed plate limiting mechanism, it is possible to make the movable bed board more stable when it is not disengaged from the bed frame, to facilitate the subsequent moving of the movable bed board to disengage from the bed frame into the medical space more smoothly, and to prevent the movable bed board from being excessively displaced when transferred out from the medical space.

FIG. 35 is a schematic diagram of the use of a medical system according to some embodiments of the present disclosure.

Referring to FIGS. 3-4, 24-26, and 35, in some embodiments, the medical system may include a medical device 1100 and a medical bed apparatus 2000, wherein the medical device 1100 may include a medical space 1110. The medical space 1110 may be a circular space around the subject. An axial direction of the medical space 1110 is the Y direction in the drawings. At least a portion of the medical bed 2100 of the medical bed apparatus 2000 may be located within the medical space 1110. In some embodiments, the medical bed 2100 of the medical bed apparatus 2000 passes through the medical space 1110 along the axial direction of the medical space 1110. By using the above medical bed apparatus 2000, the medical system can not only transport a subject requiring radiological diagnosis and treatment more conveniently and safely but also stably perform radiological diagnosis and treatment when the axial length of the medical space is relatively long.

In some embodiments, the medical system may include an imaging device and the medical device 1100, and an isocenter of the imaging device may coincide with an isocenter of the medical device 1100. The medical device 1100 may cooperate with the imaging device, allowing the medical system to have both radiographic imaging and radiation therapy functions, with radiation therapy being conducted under the guidance of radiographic imaging. The isocenters of the imaging device and the medical device 1100 refer to points around which an accelerator rotates during radiographic imaging and treatment processes. By configuring the isocenter of the imaging device to coincide with the isocenter of the medical device 1100, the accuracy of image guidance during radiation therapy can be ensured.

In some embodiments, the medical system may include a magnetic resonance imaging (MRI) device, a positron emission tomography (PET) device, a positron emission computed tomography (PECT) device, a computed tomography (CT) device, an X-ray imaging device, a single-photon emission computed tomography (SPET) device, an ultrasound device, or the like. The medical system may include a radiation therapy (RT) device. The medical system may include one of the above imaging device and treatment device or a combination thereof. For example, when multiple devices of the medical system need to scan and detect a subject, the axial length of the medical space 1110 may be relatively long (which may be considered as the accumulation of the axial length of the medical space 1110 corresponding to each medical device 1100). By supporting two ends of the medical bed board 2120 with the supporting device 2110, the subject can be more stably supported. Additionally, if the medical bed 2100 described in FIGS. 16-18 is used, the subject can be more stably supported and safely translated and rotated during movements, especially when the axial length of the medical space 2100 is relatively long.

In some embodiments, the conveying device 2300 may transport the movable bed board 2220 into at least a portion of the medical space 1110 in which the medical bed 2100 is located. In some embodiments, the medical space 1110 may be a circular space around the subject.

In some embodiments, a portion of the support device 2110 of the medical bed apparatus 2000 may be located at one end of an axial direction of the medical space 1110, and another portion of the support device 2110 may be located at the other end along the axial direction of the medical space 1110. For example, a portion of the at least three telescopic rods of the support device 2110 may be located at one end of the axial direction of the circular medical space, and another portion of the at least three telescopic rods may be located at the other end of the axial direction of the medical space 1110. With this configuration, at least a portion of the medical bed board 2120 of the medical bed 2100 is located within the medical space 1110, and the length direction of the medical bed board 2120 is parallel to the axial direction of the medical space 1110. In some embodiments, the medical bed 2100 may transport the subject into the circular medical space, and when the medical device 1100 performs scanning diagnosis on the subject, the plurality of telescopic rods of the medical bed 2100 may be controlled to extend or retract according to diagnostic and treatment requirements so as to adjust a position of the medical bed board 2120 in the medical space 1110 and a posture of the subject, thereby facilitating scanning diagnosis of the subject. In some embodiments, if the axial length of the medical space 1110 of the medical device 1100 is relatively long, a conveying distance along the length direction of the medical bed board 2120 is relatively long accordingly. By applying the medical bed 2100 to the medical system, with at least three telescopic rods supporting the medical bed board at two ends of the medical bed board along its length direction, problems such as sagging or bending of a cantilever bed plate in a cantilever-type medical bed due to lack of support at one end can be avoided, thus improving safety during a scanning detection process. Additionally, leveraging the advantages of the medical bed 2100, which has multiple degrees of freedom, higher motion control precision, and lower inertia, can meet requirements on the spatial position and posture of the subject during the scanning detection process.

Some embodiments of the present disclosure further provide a medical method, which uses the medical system described in any of the above embodiments for medical therapy or medical imaging. The medical method may include one or more of the following operations:

The movable bed board 2220 is transferred through the conveying device 2300 of the medical bed apparatus 2000, the movable bed board 2220 is placed onto the medical bed board 2120 of the medical bed 2100 of the medical bed apparatus 2000 at a position within the medical space 1110 of the medical device 1100; a position of the subject is adjusted by the medical bed 2100 by driving, using the support device 2110 of the medical bed 2100, the medical bed board to move; and a medical operation on the subject is performed using the medical device 1100.

A operation may be performed on the subject using the medical device 1100.

In some embodiments, before the conveying device 2300 transports the movable bed board 2220, the subject may be transferred to a preset position using the transfer bed 2200 of the medical bed apparatus 2000 and the movable bed board 2220 may be detached from the movable bed body 2210 of the transfer bed 2200.

In some embodiments, the subject may be transported to the preset position through other manners, for example, via a wheelchair or a regular hospital bed.

In some embodiments, after the movable bed board 2220 is transferred into the medical space 1110, the position of the movable bed board 2220 within the medical space 1110 may be further adjusted by the conveying device 2300 to modify a relative position between the subject and one or more modalities of the medical system. For example, the conveying device 2300 may adjust the subject to correspond to an imaging modality by adjusting the movable bed board 2220 in order to image the subject. As another example, the conveying device 2300 may adjust the subject to correspond to a radiation therapy modality by adjusting the movable bed board 2220 in order to administer radiation therapy to the subject.

In the above operations, the preset position may be understood as the position where a conveying mechanism (e.g., the conveying device 2300) transports the movable bed board 2220 detached from the movable bed body 2210. For example, the preset position may be a position where the conveying device 2300 docks with the movable bed board 2220 through the docking assembly 2400.

In some embodiments, the operation of detaching the movable bed body 2210 from the movable bed board 2220 may be performed after the transfer bed 2200 has moved to the preset position, thereby ensuring stable connection between the movable bed body 2210 and the movable bed board 2220 during the transfer process. In some embodiments, the operation of detaching the movable bed body 2210 from the movable bed board 2220 may be performed before the transfer bed 2200 moves to the preset position.

In some embodiments, during the process of adjusting the position of the subject through the support device 2110, an isocenter of the medical device 1100 may be positioned within a region of interest (e.g., a lesion site, a suspected lesion site, etc.) on the subject. More descriptions of other relevant aspects of the method described above may be found in the relevant descriptions of the medical bed apparatus 2000 and the medical system above, which will not be repeated here.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

What is claimed is:

1. A medical bed apparatus, comprising a medical bed, a movable bed board, and a conveying device; wherein
the conveying device is detachably docked with the movable bed board, and the conveying device is configured to transfer, on the medical bed, the movable bed board.

2. The medical bed apparatus of claim 1, wherein the medical bed includes a medical bed board and a support device, and the support device is configured to drive the medical bed board to move in multiple degrees of freedom.

3. The medical bed apparatus of claim 2, wherein the support device is configured to drive the medical bed board to move along three orthogonal directions; or
the support device is configured to drive the medical bed board to move in six degrees of freedom.

4. The medical bed apparatus of claim 2, wherein the support device includes a plurality of telescopic rods; wherein
for each of the plurality of telescopic rods, one end of the telescopic rod is connected with the medical bed board via a first hinge seat, and the other end of the telescopic rod is connected with a second hinge seat; and
a portion of the plurality of telescopic rods is connected to one end of the medical bed board, and another portion of the plurality of telescopic rods is connected to the other end of the medical bed board.

5. The medical bed apparatus of claim 4, wherein the first hinge seats are spaced apart along a first circumference, and the second hinge seats are spaced apart along a second circumference.

6. The medical bed apparatus of claim 4, wherein the support device includes at least three telescopic rods, wherein a portion of the at least three telescopic rods is connected with the one end of the medical bed board, and the rest of the at least three telescopic rods is connected with the other end of the medical bed board.

7. The medical bed apparatus of claim 4, wherein the support device includes six telescopic rods, two of the six telescopic rods are connected with the one end of the medical bed board, and the remaining four of the six telescopic rods are connected with the other end of the medical bed board; or
the support device includes six telescopic rods, three of the six telescopic rods are connected with the one end of the medical bed board, and the remaining three of the six telescopic rods are connected with the other end of the medical bed board.

8. The medical bed apparatus of claim 1, wherein the movable bed board, as a first part of a transfer bed, is detachably mounted on a movable bed body which is a second part of the transfer bed.

9. The medical bed apparatus of claim 8, wherein the conveying device is disposed on the medical bed; and
the conveying device is detachably docked with at least a portion of the transfer bed via a docking assembly.

10. The medical bed apparatus of claim 9, wherein the docking assembly includes a first docking member, a second docking member, and a locking mechanism;
one of the first docking member and the second docking member is disposed on the movable bed board, and the other of the first docking member and the second docking member is disposed on the conveying device; and
the first docking member is configured to dock with the second docking member, and the locking mechanism is configured to connect and lock the first docking member and the second docking member docked with each other.

11. The medical bed apparatus of claim 9, wherein the docking assembly includes a receiving plate and a gap, the receiving plate is disposed on the conveying device, the gap is located between the movable bed body and the movable bed board, and the receiving plate is capable of being inserted into the gap to support the movable bed board.

12. The medical bed apparatus of claim 10, wherein the first docking member includes a docking protrusion, and the second docking member includes a docking groove;
one of an outer wall of the docking protrusion and an inner wall of the docking groove is provided with a helical groove, and the other of the outer wall of the docking protrusion and the inner wall of the docking groove is provided with a protrusion part capable of being inserted into the helical groove; and
the locking mechanism includes a first driving member, and the first driving member is configured to drive the docking protrusion to rotate along an axis of the helical groove, so that the protrusion part moves along the helical groove.

13. The medical bed apparatus of claim 10, wherein
the first docking member includes at least one hook, and the second docking member includes at least one docking groove;
the at least one hook is provided on one of the conveying device and the movable bed board, and the at least one docking groove is provided on another one of the conveying device and the movable bed board; and
the at least one hook is capable of being connected with or disconnected from the at least one docking groove by rotation.

14. The medical bed apparatus of claim 13, further comprising at least one resilient member and an unlocking member; wherein
the at least one hook is disposed on the unlocking member, and the at least one resilient member is arranged between the conveying device and the unlocking member.

15. The medical bed apparatus of claim 13, further comprising a drive mechanism driving the at least one hook to rotate, wherein the drive mechanism includes a drive cable and a reel, the reel is disposed at one end of the medical bed that is far from a docking location of the conveying device and the movable bed board, and the drive cable is connected with the reel and the unlocking member.

16. The medical bed apparatus of claim 14, wherein each of the at least one hook is provided with a first guiding slope, the movable bed board is provided with a guiding member, the guide member and the at least one docking groove are arranged along a conveying direction of the conveying device, and the guide member cooperates with the first guiding slope.

17. The medical bed apparatus of claim 1, further comprising a lifting platform, wherein the lifting platform is disposed on the medical bed and is capable of being raised and lowered.

18. The medical bed apparatus of claim 17, wherein the lifting platform includes a lifting seat, a lifting drive mechanism, and a bed frame;
the bed frame is fixed to the lifting seat, the movable bed board is detachably disposed on the bed frame, and the lifting seat is connected to the medical bed; and
the lifting drive mechanism is drivingly connected to the lifting seat.

19. A medical system, comprising a medical device and the medical bed apparatus of claim 1, wherein the medical device includes a medical space, a medical bed board of the medical bed of the medical bed apparatus is configured to pass through the medical space along an axial direction of the medical space.

20. A medical method using the medical system of claim 19 for medical therapy or medical imaging, comprising:
transferring the movable bed board through the conveying device of the medical bed apparatus;
placing the movable bed board onto the medical bed board of the medical bed of the medical bed apparatus at a position within the medical space of the medical device;
adjusting a position of a subject by driving, using a support device of the medical bed, the medical bed board to move; and
performing a medical operation on the subject using the medical device.
